(19) United States
(12) Patent Application Publication
(10) Pub. No.: US 2022/0370017 A1
(43) Pub. Date: Nov. 24, 2022

(54) Personalized prediction and identification of the incidence of atrial arrhythmias from other cardiac rhythms

(71) Applicant: University of Cincinnati, Cincinnati, OH (US)

(72) Inventors: Deeptankar DeMazumder, Baltimore, MD (US); Benjamin Vaughan, Cincinnati, OH (US)

(73) Assignee: University of Cincinnati, Cincinnati, OH (US)

(21) Appl. No.: 17/745,690

(22) Filed: May 16, 2022

Related U.S. Application Data
(60) Provisional application No. 63/188,980, filed on May 14, 2021.

(51) Int. Cl. A61B 5/00; A61B 5/0205; A61B 5/024; A61B 5/318; A61K 31/7048; A61P 9/06

(52) U.S. Cl. CPC A61B 5/7275; A61B 5/0205; A61B 5/02405; A61B 5/4806; A61B 5/318; A61K 31/7048; A61P 9/06

(57) ABSTRACT

Provided herein is a method for diagnosing and treating a subject at risk for atrial fibrillation (AF) or related health conditions, the method including: collecting one or more physiological signals from the subject in a sleep state or an awake state; extracting time series data from the one or more physiological signals; performing dynamic analyses of the time series data using artificial intelligence, wherein the artificial intelligence calculates a series of dynamic measurements, said dynamic measurements being indicative of a probability of an onset of an abnormal atrial rhythm; providing an integrated personalized risk score including the dynamic measurements, wherein the integrated personalized risk score is indicative of a probability of an onset of AF in the subject; diagnosing the subject as being at risk for AF when the integrated personalized risk score exceeds a threshold value, wherein the threshold value is calculated by the artificial intelligence based on a library of stored data; and treating the diagnosed subject with an effective therapy to prevent or treat AF or AF-related health conditions.

### CROSS-REFERENCE TO RELATED APPLICATIONS

This application claims priority to U.S. Provisional Application Ser. No. 63/188,980, filed May 14, 2021, which is incorporated herein by reference in its entirety.

### STATEMENT REGARDING FEDERALLY FUNDED RESEARCH

This invention was made with government support under HK130662, HL119810, and HL157941 awarded by National Institutes of Health. The government has certain rights in the invention. This invention was also supported by the American Heart Association, funds identified as AHA 19AIML34930039 awarded by the AHA Institute for Precision Medicine.

### TECHNICAL FIELD

The present invention relates to nonlinear dynamics and predictive modeling in clinical epidemiology, namely atrial fibrillation risk stratification.

### BACKGROUND

Atrial fibrillation (AF), the most common cardiac arrhythmia, is associated with a high risk of stroke, heart failure, and premature death. Early diagnosis of AF leading to prophylactic therapy can prevent these devastating complications. However, current strategies for predicting AF incidence are limited. The risk of developing AF can be decreased with better control of hypertension, improvements in weight and exercise habits, and implementation of a robust risk prediction score. The substantial burden of AF in asymptomatic, ostensibly healthy, middle-aged, and older persons and high rate of missed diagnoses of AF until catastrophic clinical presentation motivates the design of new, more effective strategies for prediction of incident AF.

Despite considerable progress in our understanding of the underlying pathophysiology of AF, there has been relatively little progress in predicting AF incidence. The current state-of-the-art for predicting AF incidence are the CHA₂DS₂-VASc and CHARGE-AF risk scores. However, both scores have major limitations. Because their calculations are dependent on clinical history, they suffer from inaccuracies that commonly occur in electronic health records. Further, they are comprised of generalized risk factors such as those for atherosclerosis and overlap with the Framingham risk score (FRS), which predicts myocardial infarction and death. As such, these risk scores perform poorly in people with low cardiovascular risk. Importantly, they have insufficient prognostic discrimination for use in routine clinical practice.

Despite extensive research extending over several decades, relatively little progress has been made for predicting AF incidence in routine clinical practice. The majority of the extensively studied and established AF risk factors (e.g., older age, male sex, hypertension, diabetes, obesity, valvular heart disease, cardiomyopathy, alcohol, smoking, sleep disordered breathing (SDB)) overlap with the risk factors for atherosclerosis. The CHA₂DS₂-VASc was originally developed to determine stroke risk after diagnosis of AF, but has recently been used for predicting incident AF. By contrast, CHARGE-AF was derived for predicting AF incidence in the Framingham Heart Study, Cardiovascular Health Study, and Atherosclerosis Risk in Communities Study and subsequently validated in large community-based studies. However, both scores are dependent entirely on documentation available from a patient's history and, as such, suffer from the inaccuracies that occur routinely in the electronic health record. For example, the absence or presence of hypertension or diabetes can be misdiagnosed (e.g., “white coat hypertension”), undiagnosed, or missing from the history.

The FRS estimates the 10 year risk of manifesting clinical cardiovascular disease (coronary artery disease (CAD), stroke, peripheral vascular disease (PVD), congestive heart failure (CHF), cardiac death) by assigning risk points to various factors, including age, sex, HDL, total cholesterol, systolic blood pressure, smoking history, and diabetes. Based on the total point value, an individual can be classified as low, intermediate, or high risk for a cardiovascular event.

CHA₂DS₂-VASc calculates a score for stroke risk associated with AF and is used to risk-stratify individuals for clinical decisions regarding treatment. Importantly, this score is typically only used for patients who already have identified AF and typically only used in patients aged 65-74. This evaluates a variety of risk factors, including age, sex, CHF history, hypertension history, adverse events, vascular disease history, and diabetes history.

Whereas FRS and CHA₂DS₂-VASc provide ordinal variables, the CHARGE-AF score is a continuous variable. CHARGE-AF predicts incident AF risk by evaluating risk factors including age (per 5 year increment), race, height (per 10 cm increment), weight (per 15 kg increment), systolic blood pressure (per 20 mm Hg increment), diastolic blood pressure (per 10 mm Hg increment), smoking (current vs former/never), antihypertensive medication use, diabetes, heart failure, and myocardial infarction. The CHARGE-AF uses a weighted score calculated as:

$$\begin{matrix}CHARGE AF = 0.508 \times age ( 5 years ) + 0.248 height ( 10 cm ) + 0.115 \times weight ( 15 kg ) + 0.197 \\ \times systolic blood pressure ( 20 mmHg ) - 0.101 \\ \times diastolic blood pressure ( 10 mmHg ) + 0.359 \times current smoker + 0.349 \\ \times antihypertensive medication + 0.237 \times diabetes + 0.701 \\ \times congestive heart failure + 0.496 \times myocardial infarction .\end{matrix}$$

Accordingly, a need exists for a methodology for predicting AF in people with low cardiovascular risk for use in routine clinical practice.

### SUMMARY

Embodiments of the present disclosure are directed to methods and systems for diagnosing and treating a subject at risk for atrial fibrillation (AF), applying the technologies of clinical epidemiology, predictive modeling, nonlinear dynamics, and risk stratification to develop strategies for predicting AF incidence.

Specifically, the present disclosure utilizes nonlinear dynamic analysis of physiological signals, as described below, to show “complexity” during sinus rhythm, providing vital insight into subclinical physiological deterioration and adding unique prognostic value to established risk factors and conventional measures of heart rate variability. In particular, the present disclosure shows an important link between high vagal tone during early waking hours, chronotropic response during mild transient desaturation, subtle conduction abnormalities, complexity of sinus rhythm, and subclinical electrical instability of the atrium for predicting new onset AF.

Accordingly, one embodiment of the present disclosure is directed to a method for diagnosing and treating a subject at risk for atrial fibrillation (AF) or AF-related health conditions, the method comprising: collecting one or more physiological signals from the subject in a sleep state or an awake state; extracting time series data from the one or more physiological signals; performing dynamic analyses of the time series data using artificial intelligence, wherein the artificial intelligence calculates a series of dynamic measurements, said dynamic measurements being indicative of a probability of an onset of an abnormal atrial rhythm; providing an integrated personalized risk score comprising the dynamic measurements, wherein the integrated personalized risk score is indicative of a probability of an onset of AF in the subject; diagnosing the subject as being at risk for AF when the integrated personalized risk score exceeds a threshold value, wherein the threshold value is calculated by the artificial intelligence based on a library of stored data; and treating the diagnosed subject with an effective therapy to prevent or treat AF or AF-related health conditions.

Features and benefits of the various embodiments of the present invention will become apparent from the following description, which includes figures and examples of specific embodiments intended to give a broad representation of the invention. Various modifications will be apparent to those skilled in the art from this description and from practice of the invention. The scope is not intended to be limited to the particular forms disclosed and the invention covers all modifications, equivalents and alternatives falling within the spirit and scope of the invention as defined by the claims.

### BRIEF DESCRIPTION OF THE DRAWINGS

The following detailed description of specific embodiments of the present disclosure can be best understood when read in conjunction with the following drawings, wherein:

FIG. 1 schematically depicts a flowchart illustrating a system and method for diagnosing atrial fibrillation using a DeepEntropy (DE) model as well as a variety of data from a subject, according to one or more of the embodiments herein;

FIG. 2 depicts a standard wave form evaluated according to one or more embodiments described herein;

FIG. 3 depicts the matrix structure containing short line segments along subdiagonals, where the length of the line describes the duration where the two points are close to each other;

FIG. 4 is a representation of the Euclidian distance indicating that each point lies on an ellipse centered at another point with semiaxes r;

FIGS. 5A-5C graphically depict a comparison of key predictors and risk scores between the validation and derivation cohorts, including age, Framingham risk score and ECG dynamics (FIG. 5A), diastolic blood pressure, CHA₂DS₂ —VASc score and DeepEntropy (FIG. 5B), and non-HDL cholesterol, CHARGE-AF score, and Cincinnati AF score (CAFS) (FIG. 5C);

FIGS. 6A-C are a graphical illustration of the correspondence between conventional risk scores, age (FIG. 6A), ECG dynamics (FIG. 6B) and DeepEntropy (FIG. 6C);

FIGS. 7A-7C are graphical comparisons of distributions, probability densities and ROC curves between key predictors and risk scores, including age, Framingham risk score, and ECG dynamics (FIG. 7A), diastolic blood pressure, CHA₂DS₂—VASc score and DeepEntropy (FIG. 7B), and non-HDL cholesterol, CHARGE-AF score, and Cincinnati AF score (FIG. 7C);

FIG. 8 is a graphical depiction of the ROC area under the curve (AUC) at 6 and 10 years of follow-up, wherein values were greater for CAFS compared to the FRS, CHA₂DS₂-VASc, CHARGE-AF, age, and ECG Dynamics;

FIGS. 9A-9C are graphical depictions of the cumulative incidence of atrial fibrillation plotted by tertiles of each risk score or predictor for a follow-up of 8 years, where FIG. 9A depicts age and CHARGE-AF, FIG. 9B depicts FRS and ECG dynamics, and FIG. 9C depicts CHA₂DS₂-VASc and CAFS;

FIGS. 10A-10B depicts Hosnner-Lenneshow calibration plots showing the predicted vs. observed cumulative index for various risk stratification methods including age, Framingham risk score, and CHA₂DS₂—VASc score (FIG. 10A) and CHARGE-AF score, ECG dynamics, and Cincinnati AF score (CAFS) (FIG. 10B);

FIGS. 11A-11B depict the net reclassification improvement of conventional risk scores with the incorporation of ECG dynamics and/or CAFS;

FIGS. 12A-12C are graphical representations of data showing the comparison of various scoring methods from matched cohorts from the derivation group and the validation group showing the receiver operator curve (ROC) area under curve (AUC) (FIG. 12A), the continuous NRI of ECG Dynamics (FIG. 12B), and the continuous NRI of CAFS (FIG. 12C);

FIGS. 13A-C are graphical representations of data showing the comparison of various scoring methods from randomly selected and matched cohorts, showing the receiver operator curve (ROC) area under curve (AUC) (FIG. 13A), the continuous NRI of ECG Dynamics (FIG. 13B), and the continuous NRI of CAFS (FIG. 13C);

FIGS. 14A-D are graphical illustrations of the (ROC) (AUC) vs. Respiratory Distress Index (RDI) for different scoring methods at different levels of desaturation 3% desaturation (FIG. 14A), 3% desaturation or arousal (FIG. 14B), 5% desaturation (FIG. 14C), and 5% desaturation or arousal (FIG. 14D); and

FIG. 15 is a graphical illustration showing that incorporating ECG dynamics in a multivariate model comprised of the conventional risk scores improved both true positives and true negatives.

### DETAILED DESCRIPTION

The details of one or more embodiments of the presently-disclosed subject matter are set forth in this document. Modifications to embodiments described in this document, and other embodiments, will be evident to those of ordinary skill in the art after a study of the information provided herein.

Unless defined otherwise, all technical and scientific terms used herein have the same meaning as commonly understood by one of ordinary skill in the art to which the presently-disclosed subject matter belongs.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, pH, size, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used herein, a “subject” refers to a mammalian subject. Optionally, a subject is a human or non-human primate. Optionally, the subject is selected from the group consisting of mouse, rat, rabbit, monkey, pig, and human. In a specific embodiment, the subject is a human.

The terms “treat,” “treatment,” and “treating,” as used herein, refer to a method of alleviating or abrogating a disease, disorder, and/or symptoms thereof in a subject.

As discussed previously, conventional methods of risk stratification for atrial fibrillation calculates the risk of adverse events associated with AF, such as stroke, in a subject who has already been diagnosed with AF. Provided herein are methods and apparatus to use conventional monitoring to predict the risk of developing AF in a subject who has not been diagnosed with AF. Additionally, the methods provided herein allow for the detection of when AF is likely to develop in a subject.

Nonlinear dynamic analysis of physiological signals is an alternate, mechanistic approach that has been underexplored in clinical practice. Central to the underlying mechanism of electrical remodeling in AF is inflammation/oxidative stress, autonomic dysfunction, hemodynamic stress, and intracellular Ca²⁺ overload. The ensuing structural remodeling in AF is essentially a form of “tachycardia-induced” myopathy. Because AF is ultimately an electrical disturbance, closer inspection of the dynamic complexity of cardiac electrical activity should reveal clues about the likelihood that a new, out-of-synch electrical signal will occur, triggering new onset AF. Thus, instead of relying entirely on chart documentation, such as the CHARGE-AF and CHA₂DS₂-VASc risk scores, or clinical recognition of certain disorders (e.g., SDB), the present disclosure focused efforts on ECG waveform analysis.

Nonlinear dynamic ECG analysis is a robust approach for predicting AF. The measures of ECG complexity during sinus rhythm provide vital insight into subclinical physiological deterioration and add unique prognostic value to established risk factors and conventional measures of heart rate variability (HRV). In animals subjected to hemodynamic stress, the complexity measures reflected high inflammatory/oxidative stress, autonomic dysfunction, and intracellular Ca²⁺ overload early during disease onset, i.e., before changes in conventional measures of variability. Thus, nonlinear dynamic analysis of physiological signals provides unique insight into uncoupling of homeostatic signaling between organ systems that otherwise, cannot be distinguished by traditional deterministic measures.

Measures of HRV have been associated with incident AF, independent of cardiovascular risk factors. Whereas these prior studies used short-term HRV measures (e.g., 30-sec, 5-min), the fundamentally distinct DeepEntropy algorithm disclosed herein quantifies the complexity of otherwise apparently normal sinus rhythm over shorter (e.g., sub-millisecond) and longer (e.g., hours) terms, and reveals “hidden” signatures of impending electrical instability in the atrium. It was noted that DeepEntropy is not associated with sleep states, age-, gender-, and race-specific changes in HRV, or conventional risk factors. Importantly however, the electrophysiological complexity of sinus rhythm quantified by DeepEntropy during sleep predicted incident AF and added major independent prognostic value to conventional risk factors and scores. DeepEntropy was thus incorporated with other independent measures of ECG dynamics as well as conventional risk factors to construct the Cincinnati Atrial Fibrillation Score (CAFS). The findings presented herein confirm an identified link between high vagal tone during early waking hours, chronotropic response during mild transient desaturation, subtle conduction abnormalities, complexity of sinus rhythm, and subclinical electrical instability of the atrium for predicting new onset AF. Compared with other available models, CAFS incorporates measures of subclinical pathogenic mechanisms, namely arrhythmogenic substrates and triggers, as well as modulating factors and interactions in a personalized manner.

The data provided herein directly compares DeepEntropy and CAFS with the FRS, CHA₂DS₂-VASc, and CHARGE-AF risk scores for predicting AF incidence over 10 years of follow-up. The validation of this paradigm for detecting subclinical physiological deterioration based on nonlinear dynamic analysis of physiological signals during sleep has the potential for broad clinical impact.

The robust new score, CAFS, validated herein has multiple potential applications. First, improved identification of individuals at high risk for new onset AF remains a clinical and public health priority. By 2030, it is estimated that >12 million Americans will have AF. When AF is diagnosed early, the risks of stroke are mitigated by prophylactic anticoagulation. However, about 18-25% of people with AF are diagnosed with AF only after they have already suffered a thromboembolic stroke because current clinical methods for detecting AF are limited. Economic modeling indicates that appropriate use of antithrombotic prophylaxis in 50% of the 2.3 million patients per year with new-onset AF would prevent 29,232 strokes annually with a total direct cost reduction to Medicare by $2.4 billion USD. In this regard, improved identification of individuals who are at high risk for developing AF should improve clinical outcomes.

Despite the latest continuous ECG monitoring technology, brief AF paroxysms often remain undiagnosed. Even in intensive care units (ICU), which employ the latest technological advancements and the highest degree of continuous monitoring available in modern hospitals, many AF episodes elude clinical detection.

This enhanced, inexpensive, readily available, non-invasive, personalized AF risk score disclosed herein may directly address these important and unmet clinical needs by improved risk stratification of individuals who will and will not develop AF in the future, benefitting innumerable patients and rendering substantial socioeconomic impact. Although CAFS consistently outperformed established risk scores for predicting AF incidence, the reduced C-statistic in this validation (0.75) compared to derivation study (0.86) was primarily due to the lower prognostic performance of conventional risk factors used by CAFS (e.g., hypertension). Removing all predictors from CAFS except for age and DeepEntropy yielded a C-statistic of 0.75, driven primarily by DeepEntropy. These findings highlight the strong, independent prognostic value of DeepEntropy and warrant further investigation. Randomized clinical trials are needed to determine whether clinical implementation improves outcomes with acceptable cost-effectiveness.

In patients with AF, catheter ablation is an established and commonly-used surgical treatment. While catheter ablation may abolish AF episodes in the short term, many patients have recurrence of AF, ranging from days to years after catheter ablation. No method is available in contemporary clinical practice for identifying individuals who will and will not have AF recurrence. As such, the need for AF therapy after apparently successful catheter ablation remains unclear. Entirely due to the lack of an effective clinical tool for guiding post-ablation management, prophylactic treatment for AF recurrence (e.g., antiarrhythmics, anticoagulation) is prescribed empirically for varying periods (e.g., 0-12 months). As such, these treatments may be prematurely discontinued or not prescribed at all in some patients who will have AF recurrence. Conversely, many patients who will not have AF recurrence may be exposed to the risks of prophylactic treatment without deriving health benefit (e.g., proarrhythmia from antiarrhythmic medication).

Because AF can be induced in almost any healthy individual under certain clinical settings without conferring an increased risk of developing AF in the future, some people who have had only a single self-limiting episode of AF may not require treatment. For example, AF can be induced in a healthy person simply by rapid pacing of the heart that typically reverts spontaneously to sinus rhythm. However, current clinical guidelines qualify an individual for guideline-based AF treatment once a brief self-limiting episode of AF has been identified, primarily due to the lack of an effective clinical tool for predicting incident AF. Thus, clinical management of two patients who are identical in every way except for a single brief self-limited episode of AF can be the same as that of another patient with persistent lifelong AF. The inability to accurately discriminate the risk of developing AF unnecessarily exposes many patients without AF recurrence to side effects of AF therapy without deriving health benefit. Conversely, if an individual with a single brief AF episode is, in fact, at a higher risk of AF recurrence, then the new information from CAFS may impact clinical management, such as aggressive lifestyle and risk modification.

In embodiments, the system and methods provide a computationally efficient and robust prediction and diagnostic model for rapid detection of atrial arrhythmias in a subject.

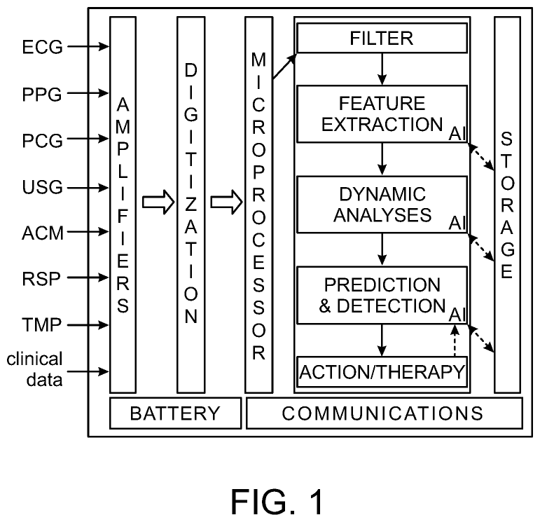

Generally, FIG. 1 depicts a principle block diagram of a system and method disclosed herein. It is noted that the method or system depicted therein may include a greater or fewer number of steps, taken in any order, without departing from the scope of the present disclosure. In embodiments, the system is capable of signal acquisition, digitization, pre-processing, analysis, detection, prediction, and treatment response.

In embodiments, the system may also store and process an individual's demographic, history and clinical data, derived parameters from physiological waveforms, baseline testing and measurements, and changes over the course of the subject's health and illness for future comparison with the same patient. In embodiments, the system may store data and analysis in a perpetual database.

Among other things, embodiments of the disclosure relate to methods, techniques, and algorithms that are intended to be implemented in a digital computer system such as generally depicted in the flowchart in FIG. 1. Such a digital computer is well-known in the art and may include at least one central processing unit and one or more memory devices. As used herein, memory devices may include, but are not limited to, RAM, ROM, hard disk, optical drives, removable drives, and the like. In embodiments, such a computer may also include auxiliary storage that can be remotely incorporated such as in a distributed computer system with distributed memory capabilities.

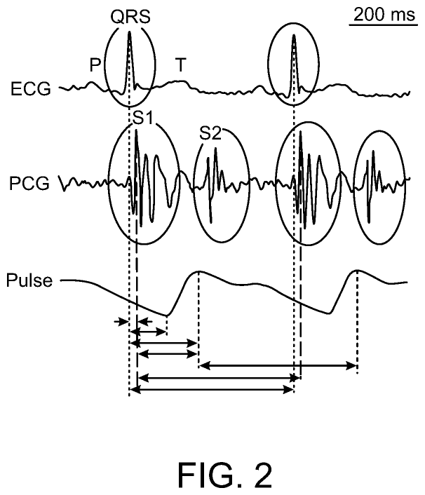

The system may further include devices or sensors for collecting one or more physiological signals from a subject. In embodiments, the physiological signals are collected via an analog circuit. In embodiments, the physiological signals collected include, but are not limited to electrocardiogram (ECG), phonocardiograms (PCG), photoplethysmogram (PPG), intra-aortic balloon pump (IABP), respiratory frequency signal prominence (RSP), transmembrane potentials (TMP), ultrasound guided (USG), and audio compression manager (ACM) waveforms. FIG. 2 depicts an illustrative waveforms of ECG and PCG signals across time intervals.

### Integrated Analysis Using DeepEntropy

In embodiments, the system can use artificial intelligence (AI) algorithms to perform nonlinear dynamic analyses on each signal or combinations of signals. In embodiments, the system may use a multi-dimensional non-linear algorithm for integrated analysis of multiple body signal dynamics, such as DeepEntropy, discussed in detail below. Whereas existing algorithms, such as CoSEn are entirely dependent on the time elapsed between two successive R waves of the QRS signal (RR intervals) for AF detection, DeepEntropy uses a multi-dimensional, non-linear algorithm for integrated analysis of multiple body signal dynamics, including but not limited to ECG, PCG, RSP, TMP, USG, ACM, and PPG waveforms. Such an integrated measure of body signal dynamics adds unique prognostic value to conventional clinical data for AF detection and prediction. Thus, in contrast to CoSEn and related algorithms, DeepEntropy incorporates the physiologically rich information on cardiac rhythm available from non-RR and non-ECG measures in contemporary monitoring devices for improved performance. Moreover, unlike CoSEn and related algorithms, DeepEntropy is not susceptible to the artifacts common to RR extraction and AF detection.

The prerequisite first step is to sort the waveform data and distinguish bonafide beat-to-beat signals from noise/aberrant signals. Then, the bonafide signals are used for feature selection (supervised and unsupervised) and to generate a risk integrator. The system can use a plurality of AI chipsets for data processing. The first AI-specific chipset discriminates the beat-to-beat ECG signals by loading and processing waveform data, performing horizontal and vertical alignment of each heartbeat and comparing each beat morphology to a signal averaged-template.

### Load and Process Waveform Data

Typical ECG interval values for a heartbeat ranges (in msec) are as follows: RR interval (400-2000); end of P to Q (10-100); duration of QR (0-100), QRS (50-150), ST (10-150) and QT (200-600). The desired data files are loaded into the system, one at a time where each file corresponds to an ECG time series for one subject. For each subject, the time series is divided into epochs (e.g., 5-mins) based on the number of samples and the sampling rate. The number of beats (beat_{1 . . . N}) in each epoch is identified by identifying the peak of the R wave for each beat (using an acceptable peak detection code).

The ECG complex for each beat is identified by including a time segment before and a time segment after the peak of the R wave of that beat. Then the time segment before the peak of the R wave is calculated as 0.25×RR interval and the time segment after the peak of the R wave is calculated as 0.75×RR interval. An algorithm subroutine can be incorporated to better calculate these dyna mic time segments before and after the R wave.

Horizontal and Vertical Alignment of each heartbeat

For each epoch, identify each beat complex and align all beats, first along the abscissa/horizontal axis (time) and then, in the ordinate/vertical axis (voltage). To do this, first calculate the mode (most frequent) ECG waveform for all the beat complexes (beat_{1 . . . N}) in that epoch and determine the time from the onset (t=0) to the time of the peak of the R wave (t=Rₚₑₐₖ) for the mode ECG waveform.

For each beat, the difference (δ) in time to Rₚₑₐₖ of that beat and time to Rₚₑₐₖ of a model ECG is calculated. For example, the δ beat δ_{N} can be calculated by subtracting the time to Rₚₑₐₖ of beat N from the time to Rₚₑₐₖ of the mode ECG waveform.

For a beat with a longer time of Rₚₑₐₖ compared to the mode ECG, the δ_{N} will be a positive time value and for a beat with a shorter time of Rₚₑₐₖ compared to the mode ECG, the δ_{N} will be a negative time value. The mean, median, standard deviation, and range of the δ value can be calculated. The system may use these calculated values to flag beats.

For each beat, the beat is horizontally aligned along the abscissa (time) axis by subtracting δ_{N} from the onset time (t=0). This will result in an initial alignment of the Rₚₑₐₖ of that beat with the Rₚₑₐₖ of the mode ECG.

Then, more refined horizontal alignment is performed using Qₚₑₐₖ. The Qwave is the sharp (positive or negative) deflection in the ECG waveform occurring just before Rₚₑₐₖ, typically in the range of 0-100 msec before Rₚₑₐₖ. Qₚₑₐₖ is identified using the first and second derivatives of the ECG segment before Rₚₑₐₖ. Each beat is horizontally aligned along the abscissa (time) axis, based on comparing the time to Qₚₑₐₖ of each beat with the mode ECG (using steps similar to those used above to align by R_{Rpeak}). In some cases, the Qwave may not be a separate deflection and instead will be identified as the steepest portion of the R wave before Rₚₑₐₖ.

After horizontal alignment is complete, the isoelectric line from the end of the P wave to just before the Q wave is used for the vertical alignment of each beat to the mode ECG, such that the vertical axis value of the isoelectric line is zero for each beat. Using a moving average, power spectral and wavelet analyses on short (e.g., 5-10 beats) and long (e.g., 30 beats) length windows of sequential beat complexes will generate dynamic QRS, P and T wave envelopes to discriminate the waveforms with >99.9% sensitivity and >99.9% specificity.

Comparing each Beat Morphology to a Signal-Averaged Template within Each Epoch

After horizontal and vertical alignments are complete with confirmation of accuracy and precision of waveform discrimination, a duplicate set of beat complexes are generated by taking the absolute value of each beat complex. An ensemble average of all the beats within each bin is generated to form a template for the original set of beat complexes (template₀) and another template from the absolute value version of the beat complexes (template_{abs}).

The X² value is calculated as the sum of difference squared for all voltage time series points for each beat compared to template₀ or template_{abs}. Thus, a X²₀ value (vs. template₀) and a X²_{abs} value (vs. template_{abs}) will be generated for each beat. In sub-segmental analyses, calculate X² values for duration of P, QRS and T waves (e.g., X²_{0-P}, X²_{abs-P}, X²_{0-QRS}, X²_{abs-QRS}, X²_{0-ST}, X²_{abs-ST})

Plots are created for each ECG record, including X²₀ and X²_{abs} values as a function of the number of beats for all beats in the entire record (all epochs), and plots of RR vs. PP, RR vs. QT, and RRₙ vs. RRₙ₊₁. Each record can be wirelessly streamed to an external device/computer for verification of outputs.

The beat-to-beat changes in the PQ QRS, QT, J/ST and PQRST segments are quantified (e.g., per 5-min bins) based on: (1) morphology [e.g., shape, amplitude]; (2) rate [faster or slower than physiological limits]; (3) rhythm [comparison to beats before and after current beat]; and (4) patterns and time course of changes compared to other beats and other conditions.

Using a combination of Bayesian, non-linear algorithms and neural network approaches, the AI-enhanced software code automatically identifies the following with >99% sensitivity and >99% specificity: (1) bona fide ECG signals (e.g., PQRST features); (2) physiological sinus beats; (3) artifactual R-wave (i.e., noise/artifact appearing as but physiologically improbable to be R-wave); (4) machine generated noise (e.g., powerline interference, white/pink noise, etc.); and (5) human generated noise (e.g., baseline shifts, mechanical, non-cardiac electrical activity). The next chipset will process the DeepEntropy calculations for short-term AF prediction whereas, external device will process for longer-term prediction.

The DeepEntropy (DE) model includes a numerical analysis method for dynamic multi-dimensional analysis of time series data for rapid, robust and accurate AF detection and prediction. This method is calculated as follows:

Consider a dynamical system of dimension F such that P≤F variables are observable. Each observable variable is denoted as vᵢ =vᵢ(t) for i=1, 2, . . . , P, and the vector function, u: [symbol] → [symbol] ^{P}, as:

$$u ( t ) = [ \begin{matrix}{v}_{1} ( t ) \\ ⋮ \\ {v}_{P} ( t )\end{matrix} ] ,$$

The values for each variable are sampled at discrete times over a window of time length w to generate the collection of time-series.

$${v}_{i} = [ {v}_{1}^{( i )} , … , {v}_{j}^{( i )} , … , {v}_{{N}_{i}}^{( i )} ] ∈ {ℝ}^{{N}_{i}} ,$$

where vⱼ⁽ⁱ⁾=vⱼ(tⱼ⁽ⁱ⁾) are the samples, tⱼ⁽ⁱ⁾ for j=1, . . . , Nᵢ are the sample times and Nᵢ is the number of samples collected for the i^{th} variable.

Assuming the sample times that coincide for each variable (though other sample times are contemplated and possible) such that t⁽ⁱ⁾=tⱼ and Nᵢ=N for all i. In this case, the vector times-series for all variables as the matrix can be written as

$$U = [ \begin{matrix}{v}_{1}^{( 1 )} & … & {v}_{j}^{( 1 )} & … & {v}_{N}^{( 1 )} \\ ⋮ & & ⋮ & & ⋮ \\ {v}_{1}^{( i )} & … & {v}_{j}^{( i )} & … & {v}_{N}^{( i )} \\ ⋮ & & ⋮ & & ⋮ \\ {v}_{1}^{( P )} & … & {v}_{j}^{( P )} & … & {v}_{N}^{( P )}\end{matrix} ] = ∈ {ℝ}^{P \times N} .$$

This matrix can be analyzed by rows (individual time-series in isolation), a subset of rows, or the entire matrix as a single, higher-dimension time series. Based on Takens' embedding theorem 23, the phase space for each variable can be constructed by forming a set of vectors uⱼ⁽ⁱ⁾ of length m

uⱼ⁽ⁱ⁾=[vⱼ⁽ⁱ⁾, v_{j+τ}_{ᵢ}_{′}⁽ⁱ⁾, . . . , v_{j+kτ}_{ᵢ}_{′}⁽ⁱ⁾, . . . , vⱼ+(mᵢ−1)τᵢ]^{T}

for j=1,2, . . . Nᵢ−(mᵢ+1)τᵢ, where mᵢ ∈ [symbol] +is the embedding dimension and τᵢ ∈ [symbol] +is the embedding lag, both for the i^{th} time-series. The vectors u⁽ⁱ⁾ represent the i^{th} time-series as a trajectory in m-dimensional space.

Again, for simplicity, assuming mᵢ=m and τᵢ=τ for i=1, . . . , P produces a sequence of M=N−(m+1)τ that embeds the P-dimensional time-series into a (P×m) space. This embedding matrix is denoted

$${U}^{( f )} = [ \begin{matrix}{v}_{j}^{( 1 )} & {v}_{j + τ}^{( 1 )} & … & {v}_{j + k τ}^{( 1 )} & … & {v}_{j + ( m - 1 ) τ}^{( 1 )} \\ ⋮ & ⋮ & & ⋮ & & ⋮ \\ {v}_{j}^{( i )} & {v}_{j + τ}^{( i )} & … & {v}_{j}^{( i )} & … & {v}_{j + ( m - 1 ) τ}^{( i )} \\ ⋮ & ⋮ & & ⋮ & & ⋮ \\ {v}_{j}^{( P )} & {v}_{j + τ}^{( P )} & … & {v}_{j}^{( P )} & … & {v}_{j + ( m - 1 ) τ}^{( P )}\end{matrix} ] ∈ {ℝ}^{P \times m}$$

The weighted distance measure between as the map is defined as d_{w}(U^{(j)}, U^{(k)}): [symbol] ^{P×m}× [symbol] ^{P×m}→ [symbol] where d_{w}(U^{(j)}, U^{(k)})≥0 and d_{w}(U⁽ⁱ⁾, U⁽ⁱ⁾)=0 implying U^{(j)}=U^{(k)}. The weighting is dynamic and chosen such that d_{w}(U^{(j)}, U^{(k)})<1 indicates U^{(j)} and U^{(k)} are considered “close” and d_{w}(U^{(j)}, U^{(k)})>1 is considered “far.”

Applying the weighted distance map to each pair of embedding matrices yields the symmetric distance matrix

$$D = [ \begin{matrix}0 & {d}_{1 , 2} & … & {d}_{1 , M} \\ {d}_{1 , 2} & 0 & ⋱ & ⋮ \\ ⋮ & ⋱ & ⋱ & {d}_{M , M - 1} \\ {d}_{M , 1} & … & {d}_{M , M - 1} & 0\end{matrix} ] ∈ {ℝ}^{M \times M} .$$

Applying the Heaviside step function

$$H ( x ) = \{ \begin{matrix}1 & if & x > 0 \\ 0 & if & x < 0\end{matrix} ,$$

to the distance matrix, generates the matrix

H=H (b 1−D) ∈ [symbol] ^{M×M},

which contains only ones and zeros, with a one indicating U^{(j)} is “close” to U^{(k)} based on the weighted distance.

The DeepEntropy Rate (DER) is defined as:

$$DeepEntropyRate = \frac{1}{{M}^{2}} {\sum }_{i = 1}^{M} {\sum }_{j = 1}^{M} {H}_{ij} .$$

In embodiments, DE can be used for both single and multi-time series analysis as demonstrated below. The computation of DER depends on several parameters. As noted in the below examples, the optimal values of the embedding dimension, m, the embedding lag, τ, and the dynamic tolerances, r ∈ [symbol] , used to weigh the distance functions over the window w, must be determined using the mixed continuous/integer optimization problem. While smaller values of m will be less biased and result in more template matches, a higher value of m will more accurately reveal the complex dynamics of the data.

For example, in a time series with an underlying deterministic structure, increasing them will increase the conditional probability of a forward match because conditioning on a longer vector increases predictive accuracy. By contrast, noise from measurement errors or system interactions may not permit longer templates to match as m is increased, resulting in fewer matches. Beyond a certain length, most m-dimensional templates will be identified based on chance rather than similarity and (m+1) dimensional matches will be so few that the statistics become unreliable.

Conversely, for a value of m that is too small, there will be too many (m+1) dimensional template matches, and not enough predictive information will be contained in the short template leading to an underestimation of the probability of a forward match. For small to moderately large embedding dimensions m (e.g., m=1, 2, . . . , 20), we can calculate the conditional probabilities that two matrices of length m that match within the dynamic threshold r will also continue to match (d_{w}<1) at the next point m+1.

Choosing the most appropriate r is as essential for determining the dynamics as selecting the optimal m and τ. For example, if the value of r is too small (e.g., smaller than the typical noise amplitude), then many matrices that are in fact similar will fail to match. Conversely, if r is too large, there will be a loss in discriminating power simply because most m-dimensional templates will look similar to one. Thus, the current invention uses an iterative machine learning component to vary r as a function of the signal noise to obtain the most accurate measure of the dynamics.

### DE Analysis of a Single Time Series

For a single row selected from Uⱼ, a single time series is provided. As an example, the computation of DE from the RR interval time series will be described.

A set of vectors uⱼ of length m are formed as

u^{(j)}=(RRⱼ, RR_{j+τ}, . . . , RR_{j+kτ}, . . . , RR_{j+(m-1)τ})

For j=1, . . . , M, where M=N−(m+1)τ, N is the number of samples in the time series, m is the embedding dimension, and τ is the embedding lag. The u^{(j)} vectors represent the RR interval time series as a trajectory in m dimensional space.

The weighted distance function is taken as the scaled Euclidean distance function squared with a dynamic threshold, r, as the weight:

$${d}_{r} ( {u}^{( j )} , {u}^{( k )} ) = \frac{1}{r} {‖ {u}^{( j )} - {u}^{( k )} ‖}_{2}^{2} = \frac{1}{r} {\sum }_{i = 1}^{M} {( {u}_{i}^{( j )} - {u}_{i}^{( k )} )}^{2} .$$

If d (u^{(j)}, u^{(k)})<1, the vector u^{(k)} lies within the m-dimensional hypersphere of radius r centered at u^{(j)} and u^{(k)} is described as close to u^{(j)}. Applying the Heaviside function to the symmetric distance matrix,

Dᵢⱼ=dᵣ (u⁽ⁱ⁾, u^{(j)}),

results in the matrix: H=H(1−D) ∈ [symbol] ^{M×M}.

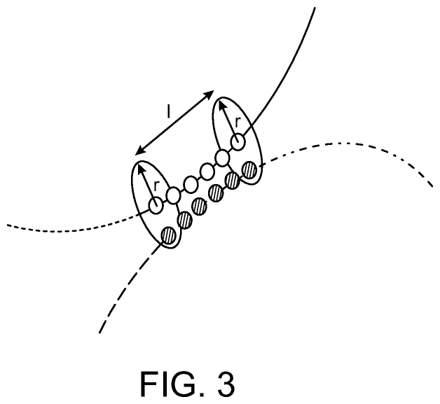

The matrix structure typically contains short line segments of ones along the subdiagonals. The lengths of these diagonal lines describe the duration where the two points are close to each other (depicted in FIG. 3). The length of these lines are directly related to the ratio of predictability inherent to the system.

The DER quantifies the matrix by a ratio of ones and zeroes for a given m, r, and τ.

After excluding diagonal lines formed by the tangential motion of the trajectory using a minimum value threshold (lₘᵢₙ), the statistical moments of the lengths of the remaining diagonal lines reveals the dynamic properties of the time series. For example, the inverse of the maximum length of the diagonal lines, lₘₐₓ, correlates with the largest positive Lyapunov exponent. The average diagonal line length, lₘₑₐₙ is obtained as

$${l}_{mean} = \frac{{\sum }_{l = {l}_{min}}^{{l}_{max}} {lN}_{l}}{{\sum }_{l = {l}_{min}}^{{l}_{max}} {N}_{l}}$$

Where Nₗ is the number of length l lines at the embedding dimension, m, the embedding lag, τ, and the dynamic tolerances, r ∈ [symbol] ^{PP}, used to weigh the distance functions over a window of time length w. Each l where Nₗ>1 is defined as a template.

To obtain an accurate measure of the dynamics of the time series, the optimal values of m, τ, w, and r are determined using a mixed continuous/integer optimization problem:

Maximize (m, τ, w, r)

where m, τ, and w are positive integers and r is a vector of real numbers with the simple bounds rᵢ>0 for i=1, . . . , P. The objective function, f, depends on the number of matches for each repeating template to calculate the conditional probability that the templates will continue to match for increasing values of m and/or τ by varying the dynamic tolerances to yield a minimum number of matches for the templates within the window w. Additionally, f should prefer larger m and smaller elements r relative to the signal noise and statistical moments of the time series. This optimization for the parameters can be done for the entire dataset or performed dynamically as new data are received.

### DE Analysis of Multiple Time-Series

Selecting more than one or all the individual time series, a vector time series is described by the matrix

$$U = [ \begin{matrix}{v}_{1}^{( 1 )} & … & {v}_{j}^{( 1 )} & … & {v}_{N}^{( 1 )} \\ ⋮ & & ⋮ & & ⋮ \\ {v}_{1}^{( i )} & … & {v}_{j}^{( i )} & … & {v}_{N}^{( i )} \\ ⋮ & & ⋮ & & ⋮ \\ {v}_{1}^{( P )} & … & {v}_{j}^{( P )} & … & {v}_{N}^{( P )}\end{matrix} ]$$

where each row denotes a selected time series, v⁽ⁱ⁾, whose elements are sampled at times tⱼ for j=1, . . . , N. By selected a subset of the elements of the matrix U, we obtain the (P×m)-dimensional embedding

$${U}^{( j )} = [ \begin{matrix}{v}_{j}^{( 1 )} & {v}_{j + τ}^{( 1 )} & … & {v}_{j + k τ}^{( 1 )} & … & {v}_{j + ( m - 1 ) τ}^{( 1 )} \\ ⋮ & ⋮ & & ⋮ & & ⋮ \\ {v}_{j}^{( i )} & {v}_{j + τ}^{( i )} & … & {v}_{j}^{( i )} & … & {v}_{j + ( m - 1 ) τ}^{( i )} \\ ⋮ & ⋮ & & ⋮ & & ⋮ \\ {v}_{j}^{( P )} & {v}_{j + τ}^{( P )} & … & {v}_{j}^{( P )} & … & {v}_{j + ( m - 1 ) τ}^{( P )}\end{matrix} ] ,$$

for j=1, . . . , M, where P is the number of selected time series, M=N−(m+1)τ, N is the number of samples in the time series, m is the embedding dimension, and r is the embedding lag.

There are many choices of weighted distance functions that map a pair of embeddings to a scalar. For simplicity, a distance function analogous to the Euclidian distance was chosen as an example, but other functions are contemplated and possible. The difference matrix is defined as ^{(j,k}=U^{(j)}−U^{(k)}.

The columns of ^{(i,k)} denote the difference between the P-dimensional vector of the value of each signal at a specific time. Partitioning the difference matrix by columns for each column difference vector

Δ^{(j,k)}=[δ₁^{(j,k)}, . . . , δ_{c}^{(j,k)}, . . . , δₘ^{(j,k)}]

allows for the computation of the weighted Euclidian distance

$${‖ {δ}_{c}^{( j , k )} ‖}_{2 , r}^{2} = {\sum }_{i = 1}^{P} {( \frac{{Δ}_{i , c}^{( j , k )}}{{r}_{i}} )}^{2} = {\sum }_{i = 1}^{P} {( \frac{{U}_{i , c}^{( j )} - {U}_{i , c}^{( k )}}{{r}_{i}} )}^{2} .$$

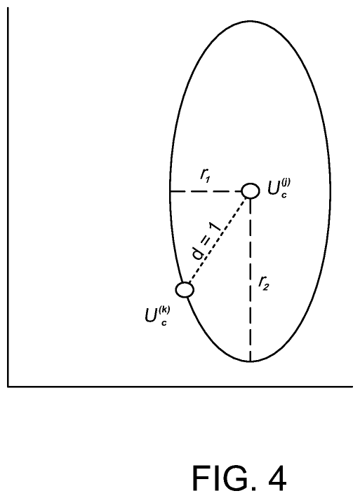

When this weighted Euclidean distance is one, the c^{th} column of U^{(k)} lies on the axis-aligned hyper-ellipse centered at the c^{th} column of U^{(j)} with semi-axes defined by the vector r=[r₁, . . . , rₚ]^{T}. For P=2, a distance of one indicates that each point lies on an ellipse centered at the other point with semi-axes r, as depicted in FIG. 4.

The weighted distance allows the system to compensate for the varying scales across different types of signals. The final weighted distance measure is the sum of the squared column distances:

$${d}_{w} ( {U}^{( j )} , {U}^{( k )} ) = {\sum }_{c = 1}^{m} {‖ {δ}_{c}^{( j , k )} ‖}_{2 , r}^{2} = {\sum }_{c = 1}^{m} {\sum }_{i = 1}^{P} {( \frac{{U}_{i , c}^{( j )} - {U}_{i , c}^{( k )}}{{r}_{i}} )}^{2} .$$

As described in the single time series analysis, the weighted distance map is applied to each pair of embedding matrices to yield the symmetric distance matrix D. The Heaviside step function is then applied to generate the matrix H=(1−D), which contains only ones and zeros, with a one indicating U^{(j)} is “close” to U^{(k)} based on the weighted distance.

As described in the single time series analysis, the statistical moments are calculated and an accurate measure of the dynamics of the time series is obtained using a mixed continuous/integer optimization problem.

In embodiments, the system can amplify, digitize, and precisely and selectively filter analog waveforms, such as the data collected from a patient as discussed herein. In embodiments, the system can be integrated with DE as discussed above to perform these functions. For example, the signal waveform time series may be transformed into multiple parallel time series using absolute, square, integral, differentiation, or other functions to facilitate feature extraction. In embodiments, the system is capable of discriminating the signal from noise and/or artifacts. In embodiments, discrimination of bona fide signals from noise/artifact for extremely precise feature extraction is achieved using various analyses, including, but not limited to: Fast Fourier Transform, power spectral density, peak detection, signal-averaging, template matching, envelopes, and moving averages.

As an illustrative example, the output signals from an analog circuit (e.g., ECG, PCG, PPG, USG) can be digitized with a multi-channel, 24-bit analog-to-digital converter (e.g., Texas Instruments ADS131E06) at sampling rates of 1-1000 Hz with a 32-bit microcontroller (e.g., Atmel ATSAM4S16C), which can filter, store and transmit data via USB or Bluetooth connection (e.g., Texas Instruments LMX9838). The feature extraction, dynamic analyses, and risk prediction can be performed by a computationally efficient central processing unit (e.g., next-generation Snapdragon, Qualcomm).

Illustrative, but non-limiting examples of the data that can be evaluated by the system are further discussed herein. An ECG (electrocardiogram) is a robust signal that reflects the electrical properties of the heart. The ECG can also be used as a fiducial time reference marker for other physiological measurements that are directly or indirectly dependent on cardiovascular function. Whereas typical ECG analysis algorithms for AF focus on the RR interval, in embodiments, the system can perform integrated measures of the RR as well as other intra-beat intervals (e.g., PR, QRST). For example, in embodiments, the system can perform an integrated measure of atrial and ventricular conduction abnormalities for AF risk prediction. Under typical analysis, in the absence of symptoms, intra-atrial or atrioventricular conduction delays, such as first-degree heart block (i.e., prolongation of the ECG's PR interval) or intraventricular conduction delays (e.g., incomplete right bundle branch block) are not considered to have clinical significance. Such delays are typically labeled as a benign finding in contemporary clinical practice. As such, only gross, low-resolution measures of these conduction delays are obtained in routine clinical practice. However, integrated measures of intra-atrial, atrioventricular and intraventricular conduction changes during apparently normal sinus rhythm is a major independent predictor of future AF in people within certain demographics, history and physiological states. Further, dynamic integrated analysis of high-resolution recordings of these conduction delays can increase prognostic value of other data.

PPG (photoplethysmography) is widely used to determine the blood hemoglobin oxygen saturation. Moreover, it can be used as an additional fiducial time reference marker to compare changes in cardiovascular pulse wave and electrical properties of the heart for an integrative electromechanical assessment of the heart. For example, the PPG waveform reflective system (e.g., wavelength of ˜940 nm) is sensitive to blood volume flow within the illuminated region. A phonocardiogram (PCG) is a specialized sound waveform that can measure low and high frequency components of the heart sounds, which reflect mechanical activities during each heartbeat cycle. The heart sounds also serve as an additional fiducial time reference marker. For example, the first heart sound (S1) can be used as a time reference for the opening of the aortic valve, the starting point of the PPG waveform, or another arterial (e.g., IABP), venous, or arteriovenous waveform.

In embodiments, RSP waveforms also add important information. For example, RSP derived parameters provide insight into different degrees of underlying lung disease (e.g., restrictive, obstructive) that, in certain individuals, can increase the risk of atrial arrhythmias such as atrial tachycardia, premature atrial contraction, and AF. In embodiments, RSP derived parameters such as the proportion of the lung's forced vital capacity (FVC) that a person is able to expire within a second of forced expiration (FEV1) strongly predicts the incidence of AF.

The ACM waveform may be used by an advanced AI algorithm to “learn” the different signals relative to a person's position, movement and state (e.g., awake, sleeping, running). Moreover, USG technology is rapidly evolving into small portable devices that can add unique prognostic value. For example, the M-mode USG provides a 1D view of the heart at a very high time resolution when aligned perpendicular to structures of interest.

In embodiments, any of the sensors or apparatus for capturing the physiological signals can be incorporated into a device configured to capture and transmit the data. In embodiments, the device can incorporate DeepEntropy or other AI to preform-nonlinear analysis on the collected data. The device can be implantable, non-implantable, or an adjunct to another existing device. In embodiments, the device can be incorporated as an implanted loop monitor. In embodiments, the device can be incorporated into “smart” devices, such as watches, phones, headphones and the like. In embodiments, the device can be incorporated into a “smart” bed, designed to capture data while a subject is asleep. In embodiments, the device can be incorporated into medical monitors in hospitals, ambulances, and the like. In embodiments, the device may be a separate device and be communicatively coupled to an existing device to provide input and output data.

As an illustrative example, an implantable device can be structured as a thin rectangular sheet inserted subcutaneously as a roll that spontaneously unfolds under the skin to provide electrical signal vectors. In embodiments, a non-implantable device could be an external skin patch or another form of wearable monitoring.

As previously discussed, integrated analysis of ECG adds a unique prognostic value. This is also true for integrated measures of features across different waveforms. For example, in embodiments, the system can perform an integrated measure of ECG-derived heart rate responses and PPG-derived SpO2 for AF risk prediction. Such analysis has identified that blunted heart rate responses to reduced SpO2 strongly and independently predict AF incidence in people with certain demographics, history and physiological states. Further, dynamic integrated non-linear analysis of different physiological waveforms (e.g., ECG, PPG) can increase the prognostic value beyond integrated analysis of features derived from the different waveforms.

In embodiments, integrated analysis of TMP derived data can further refine such prognostic values. For example, the differential diagnosis for the presence of higher temperature (e.g., fever), lower PPG-derived pulse oxygen saturation (SpO2), and cough (producing distinct patterns in RSP and PCG that are identified in a personalized way by the AI system in the current invention) includes pulmonary infections such as pneumonia, which increases the risk of atrial arrhythmias in people with certain demographics and medical history in certain settings. The TMP has many common applications but, in the context of arrhythmias, can add useful insight (e.g., complications of Brugada syndrome is often triggered by fever). Higher temperatures also occur in non-disease settings such as exercise or heavy exertion that would be identified by the AI system.

In performing nonlinear dynamic analysis on patients undergoing home poly-somnography (PSG), the complexity measures calculated by DeepEntropy were independent of sleep state, sleep disturbed breathing (SDB), and age-, sex-, and race-specific changes in HRV. However, the complexity of sinus rhythm indexed by DeepEntropy, strongly predicted AF incidence, independent of a comprehensive set of conventional ECG and PSG measures and established AF risk factors and scores.

In embodiments, the system can set threshold values to categorize patients as at risk for developing AF. The formula and thresholds can be tailored to specific populations or risk factors as appropriate. In embodiments, the system categorizes the diagnostic criteria based on the sensitivity and specificity of the scoring method compared with the probability, and generates a plot based on a library of data. This plot is used as a comparison for collected data. In embodiments, the plot is based on DeepEntropy. In other embodiments, the plot is based on the CAFS. If a patient scored above the threshold value based on the plot, then the patient is diagnosed as at risk for developing AF. In embodiments, the threshold value is calculated using the library of data to determine a first tertile, a second tertile, and a third tertile, wherein a score in the third tertile corresponds to an increased probability of developing AF.

In embodiments, an increase of 1 of a hazard ratio for the third tertile compared to the first tertile (t3:t1) of CAFS in a subject is indicative of an approximately 8 times greater likelihood, or odds, of the subject developing AF.

The system may also include communications interfaces to allow the system to operate as a distributed computer system. Non-limiting examples of computer interfaces that may be used include, LAN network adapters, WAN network adapters, wireless interfaces, Bluetooth interfaces, modems and other networking interfaces, though any acceptable interface is contemplated and possible. The system may also include other components that allow for communication of the components of the system and between systems. In embodiments, the system could classify statistical dynamic parameters and store selected data in a database where it can be organized and categorized across subgroups of individuals with similar histories and backgrounds and updated and analyzed using the AI algorithms.

In embodiments, the system and methods collect multimodal spatiotemporal physiological signals in said subject at a first time period. In embodiments, the system may repeat collecting physiological signals, identifications, and calculations in the subject at multiple times. In such embodiments, the system can perform statistical comparisons between the different times and prior records, including comparisons within and between said spatiotemporal signals and derived parameters.

In routine clinical practice, atrial flutter (often considered a “cousin” of AF) confers similar health risks and requires similar clinical treatment as AF, such as prophylactic anticoagulation for stroke prevention. Whereas the rhythm of AF is typically disorganized (i.e., “irregularly irregular”), the rhythm of atrial flutter is highly organized and regular. Moreover, some forms of AF can become organized and less irregular. In contrast to algorithms like CoSEn that are not designed for detecting atrial flutter, in embodiments, the system can detect both AF and atrial flutter.

In embodiments, the system can be used to create multi-dimensional prediction models for subjects. In embodiments, the system can also determine the absolute and relative probability of the onset of an abnormal atrial rhythm (e.g., atrial fibrillation) and related adverse effects. In embodiments, these predictions can determine the likelihood of events occurring within different time periods, e.g. seconds, minutes, hours, days, weeks, months, and years. In embodiments, the system may categorize the time periods as acute, subacute, or extended periods. Other appropriate classifications of time periods are contemplated and possible.

In embodiments, the system may be used for prediction and rapid detection of AF onset. Whereas CoSEn and related algorithms for cardiac rhythm discrimination only detect AF after the AF rhythm has sustained for a minimum number of heartbeats, the system may predict the onset of AF before it develops in the acute (e.g., a few seconds to minutes), subacute (e.g., days) and longer or extended (e.g., months to years) periods. As a result, the system can be used to prevent catastrophic clinical complications such as stroke. In embodiments, after AF has been predicted, a subject can be prophylactically treated with established therapies such as beta blockers, calcium channel blockers, digoxin, anti-arrhythmic medications, anticoagulants, and the like. In embodiments, a subject may be treated with experimental therapies as determined by a physician.

In embodiments, the system responds to the calculated probabilities to determine a personalized treatment plan based on a personalized assessment of the risks, benefits, and safety of the possible treatment plans, which include but are not limited to the delivery of electrical (e.g., overdrive pacing) and/or pharmacological therapy (e.g., beta blocker) for terminating or preventing the initiation of an abnormal rhythm. In embodiments, the system can advise a pre-designated health care system associated with the subject of a necessary action such as initiation of anticoagulation therapy to mitigate risks of adverse health events. In embodiments, the system can provide strategies to minimize the risk of the subject developing AF, including contacting a healthcare provider such as a cardiologist. In embodiments, the system can advise: aggressive risk factor modification, closer monitoring, electrical pacing, pharmacological therapy, surgery, and screening for and treatment of other underlying disorders. In embodiments, the treatment plans can also include life style changes, cognitive-behavioral therapy, biofeedback therapy, vasovagal maneuvers, autonomic modulation, electrical cardioversion, electrical pacing, pharmacological therapy, ablation, surgery, combinations thereof, and the like.

Illustrative, non-limiting examples of beta-blockers include atenolol, betaxolol, bisoprolol, carvedilol, esmolol, labetalol, metoprolol, nadolol, pindolol, propranolol, sotalol, timolol, carvedilol, and the like, and combinations thereof. Illustrative, non-limiting examples of calcium channel blockers include amlodipine, diltiazem, felodipine, isradipine, nicardipine, nifedipine, nisoldipine, verapamil, and the like, and combinations thereof. Illustrative, non-limiting examples of anti-arrhythmic medications include amiodarone, flecainide, ibutilide, lidocaine, procainamide, propafenone, quinidine, tocainide, and the like, and combinations thereof. Illustrative, non-limiting examples of anticoagulant medications include aspirin, a pixaban, dabigatran, rivaroxa ban, warfarin, clopidogrel, prasugrel, ticagrelor, dalteparin, edoxaban, enoxaparin, fondaparinux, idarucizumab, cilostazol, dipyrida mole, eptifibatide, prasugrel, heparin, tirofiban, vorapaxar, and the like, and combinations thereof.

In embodiments, the system can predict AF without a subject having sleep-disordered breathing. Whereas mainstream research focuses on the association between sleep-disordered breathing and AF, the system and methods identified herein can identify physiological dynamics during sleep that detect and predict AF regardless of sleep-disordered breathing.

In embodiments, the system can improve the accuracy and precision of AF detection and predictions. Whereas other algorithms use the same parameters in all individuals, in embodiments, the system can employ several AI learning components to optimize parameters for each individual. In embodiments, the system provides dynamic analysis of each body signal and combinations of body signals, stores the results in a personalized dictionary of statistical dynamic parameters for said individual as well as sub-groups of individuals with similar medical history and background, and performs statistical comparisons between the different times and prior records, including comparisons within and between spatiotemporal signals and dynamic parameters.

In embodiments, the system can determine the response to initiated therapies for atrial arrhythmias. In embodiments, the system can calculate modifications to prediction models, absolute risk, relative risk and treatment plan to incorporate AI-based learning components that tailor each predictive model to an individual. In embodiments, the system can calculate modifications to prediction models across similar groups of individuals to progressively improve predictive ability. In embodiments, the AI-learning system can guide clinical management.

Personalized action and treatment plans for preventing the onset of AF may be developed by the system. Because the chances of restoring normal sinus rhythm are the greatest with early treatment of AF, embodiments described herein may confer particular advantages in this regard. For example, an implanted device could implement automated pharmacological control of the heart rate (e.g., beta blocker such as metoprolol). In embodiments, a device may control rhythm (e.g., electrical cardioversion, pharmacological cardioversion) immediately upon or even before the onset of AF. In embodiments, devices could implement multiple therapies or strategies prophylactically or at the onset of AF. Thus, in addition to the ability to detect and terminate AF very quickly, such a device could prevent the onset of AF via early treatment using established therapies. Moreover, early prophylactic treatments such as anticoagulation therapy (e.g., aspirin, coumadin, apixaban) could prevent catastrophic clinical complications such as stroke.

In embodiments, the system can assess the need for AF therapy and discriminate between patients who are at an increased risk of future episodes of AF. Because AF can be induced in any individual under certain clinical settings without conferring an increased risk of future AF, many people who have only a single episode of AF may not require treatment. For example, AF can be induced in a healthy person simply by rapid pacing of the heart. However, current clinical guidelines qualify an individual for guideline-based AF treatment once a brief self-limiting episode of AF has been identified, primarily due to the lack of an effective clinical tool for predicting the risk of developing AF. Thus, clinical management of two patients who are identical in every way except for a single brief self-limited episode of AF can be the same as that of another patient who has persistent lifelong AF. The inability to discriminate the risk of future AF unnecessarily exposes patients without AF recurrence to the side effects of AF therapy without deriving any health benefit. Conversely, if an individual with a single brief AF episode is in fact at a higher risk of AF recurrence, that person would benefit from prophylactic therapy. In embodiments, the system directly addresses these critically important clinical needs by precise risk stratification of individuals who will and will not develop AF in the future.

The advanced automated system described herein uses artificial intelligence algorithms to “learn” which parameters for diagnosis and treatment responses are more effective for repeated events. In embodiments, these parameters can be tailored to an individual or sub-group of similar individuals to achieve the highest levels of personalized information extraction and precision therapy. Illustrative examples include catheter ablation, which is a n established and commonly-used surgical treatment for AF. While catheter ablation may abolish AF episodes in the short term, many patients have recurrence of AF, ranging from days to years after catheter ablation. I n embodiments, the system can discriminate between individuals likely and not likely to have AF recurrence, thereby providing and effective clinical tool for guiding post-ablation management, namely prophylactic treatment for AF recurrence.

### EXAMPLES

The present disclosure will be further described in the following examples, which do not limit the scope of the claims.

### Example 1

### Multivariate Analysis

Detailed, comprehensive, and systematic analysis of available clinical, ECG, and polysomnography parameters was performed on both a derivation and a validation cohort. The derivation cohort was based on a multicenter, prospective, community-based observational study of cardiovascular and other consequences of sleep-disordered breathing. The validation cohort was comprised of adults undergoing home PSG. A variety of criteria was used to evaluate both cohorts, including demographics, medical history, social history, prescribed medications, physical exam findings, laboratory results, PSG parameters, conventional ECG analyses (e.g., PQRST intervals, ST and T wave changes, time and frequency domain measures of H RV stratified by sleep state as well as independent of sleep states), established AF risk scores (FRS, CHA₂DS₂-VASc, CHARGE-AF), and complexity analyses of sinus rhythm.

In particular, established ECG measures (e.g., PQRST intervals, ST changes, established time, frequency, and nonlinear domain measures of HRV such as detrended fluctuation analysis, sample entropy, coefficient of sample entropy, Entropy X) were analyzed along with DeepEntropy. The ECG analyses were performed in 30- and 300-second epochs stratified by sleep state (e.g., rapid or non-rapid eye movement, awakenings during sleep, before or after sleep onset, 5 mins before or after waking up from sleep without falling asleep again), as well as in 1-hour epochs at specific times independent of sleep state (e.g., first hour of sleep onset, second hour, third hour, and so on).

All available clinical, ECG, and PSG measurements were input into logistic regression models with the incidence of new-onset AF labeled as an outcome of 1 and used as events to be predicted, and the rest were labeled as outcome 0. Standard maximum likelihood estimation was used to determine the coefficients for the logistic regression model, correcting both for unequal variances and correlated responses from individual patients. Whereas the estimates of regression coefficients and other model parameters were obtained in a standard fashion, the P values were corrected using the Hubert-White cluster robust standard errors, an extension of robust standard errors to deal with unequal error variance (heteroskedasticity).

In multivariate analyses, the major independent predictors of AF incidence were identified. These included conventional risk factors such as: (A) age, years; (B) history of hypertension; (C) non-high-density (non-HDL) lipoprotein-cholesterol, mg/dL; (D) diastolic blood pressure, mmHg. In addition, this calculation also included new measures of ECG dynamics during sleep including (E) chronotropic response during transient mild (<95%) hypoxemia, bpm; (F) subtle atrioventricular and intraventricular conduction dynamics; (G) high frequency component of ECG power spectral density reflecting primarily parasympathetic tone during final waking from sleep in the early morning hours, %; and (H) complexity of sinus rhythm as indexed by DeepEntropy. These independent predictors were incorporated into a multivariate model referred to as the Cincinnati AF Score (CAFS). Other variables were evaluated but did not affect the scoring. The derived and validated calculation was:

CAFS=(0.11×A)+(0.73×B)−(0.0063×C)−(0.016×D)−(0.031×E)+(0.65×F)+(0.012×G)+(0.12H)+1.

The raw ECG data were manually inspected for ectopy, noise, and other artifacts by 10 independent reviewers with >90% overlap to assess interobserver variability. Custom software developed in Matlab and Python were used to visualize, analyze and process the data. The presence of hypertension was assessed using the JNC VI criteria wherein subjects were classified as normal (<130/<85 mm Hg); high-normal (130-139/85-89 mm Hg); stage 1 hypertension (140-159/90-99 mm Hg); or stage 2 or greater hypertension (≥160/≥100 mm Hg). The presence of diabetes was assessed using the American Diabetes Association fasting criteria of a hemoglobin A1c≥6.5%. Obesity was assessed using body mass index of ≥30 kg/m². Sleep disordered breathing (SDB) was assessed by an overall respiratory distress index at 4% desaturation occurring at ≥5 events/hour.

The baseline characteristics were compared between both cohorts. The results discussed herein provide continuous variables with normal distributions are presented as mean and standard deviation, and with non-Gaussian distribution are presented as median and interquartile range. Categorical variables are presented as numbers and percentages. The performance of DeepEntropy and CAFS were directly compared with the FRS, CHA₂DS₂-VASc, and CHARGE-AF scores. Because CHA₂DS₂-VASc is a categorical variable, DeepEntropy was used in all calculations as a categorical variable.

Multivariate penalized logistic regression analyses was performed. Hazards ratios from multivariate Cox proportional analyses were adjusted. Additionally, the data underwent net reclassification improvement (NRI), and receiver operating characteristic (ROC) area under the curve (AUC) for the primary outcome of AF incidence. Ten-fold cross-validations were performed to avoid model overfitting.

The cumulative incidence of AF was estimated. No competing risks from death were identified. Proportional hazards regression was performed separately for each risk score and estimated AF risk. The Wald X₂ statistic was used to test the null hypothesis that the risk score was not associated with AF. Discrimination using the C-statistic was examined. Additionally, Cox-Snell, Schoenfeld, and scaled Schoenfeld residuals were used to rule out the possibility of violation of proportionality assumption.

Calibration plots were to compare observed binary outcomes of AF within groups of observation windows for each risk score to the predicted risk among the same follow-up windows, conditional on the vector of the risk scores and confounding exposures. Participants were divided into groups based on the predicted probability of AF obtained from a risk prediction model. The sum of expected probability within each group was calculated. Additionally, the Hosmer-Lemeshow (HL) X² statistics were calculated, which yielded low X² values, indicating good calibration.

The subgroups described herein were exclusive to the calibration analysis. These dichotomous subgroups were used to evaluate whether the predicted risks of AF were dependent on conventional risk factors (e.g., median age, gender, hypertension, obesity, sleep disturbed breathing). Further, analyses of interaction, including these variables as interaction terms in these models was performed. Age was tested as a continuous variable as well as a dichotomous variable based on the median age (i.e., <67 or ≥67 years).

All statistical analyses were performed using STATA version 17.0 (StataCorp LP, College Station, Tex., USA). Continuous variables were compared using a 2-sided t-test, and categorical variables were compared using the X² test. A p-value <0.05 was considered statistically significant unless otherwise indicated.

### Example 2

### Comparison of the Validation and Derivation Cohorts

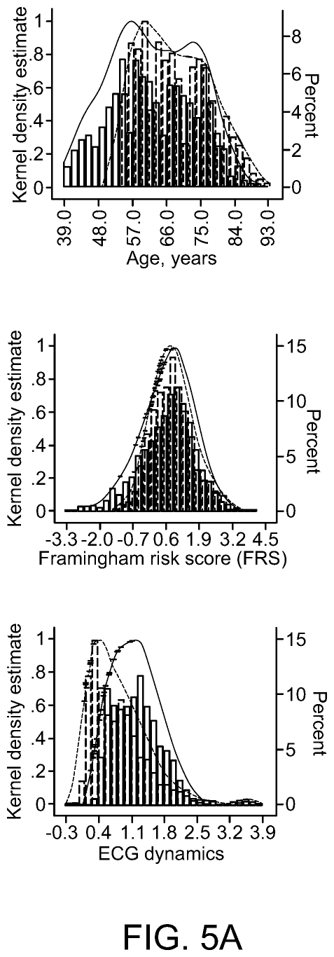

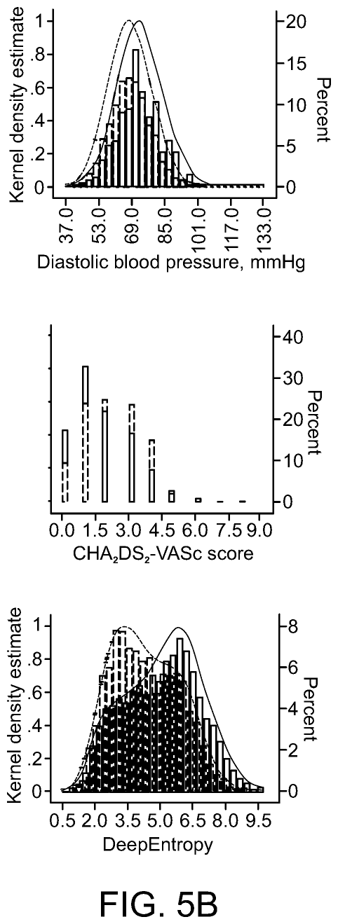

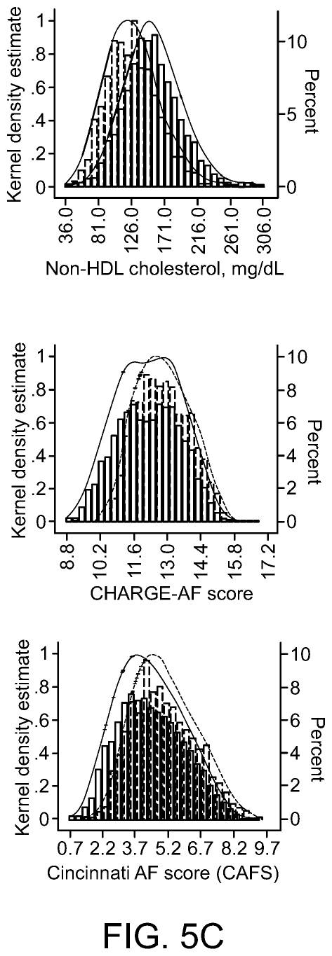

The distribution of the values for the risk predictors and scores were distinct between cohorts. FIGS. 5A-5C depict a graphical comparison of key predictors and risk scores between the validation and derivation cohorts.

The histograms depicted are superimposed with non-parametric kernel density plots, which estimate the probability density function of the predictors in each cohort. Compared to the derivation cohort, the FRS, CHA₂DS₂-VASc, CHARGE-AF and CAFS values were larger, despite the markedly lower incidence of AF (4.1 vs. 11.2%) and other cardiovascular events in the validation cohort, as demonstrated in Table 1.

TABLE 1

| | | |
|---|---|---|
| | Derivation | Validation |
| Covariate | (N = 2807) | (N = 2004) |

| | | |
|---|---|---|
| Age | 62.6 ± 11 | 68.5 ± 9.2 *** |

| | | | | |
|---|---|---|---|---|
| Female, % (N) | 54.8 | (1537) | 53.3 | (1069) |
| White, % (N) | 85.7 | (2407) | 36.3 | (728) *** |
| Black, % (N) | 7.4 | (208) | 27.9 | (559) *** |
| Hypertension, % (N) | 37.3 | (1048) | 56.4 | (1130) *** |
| Diabetes Mellitus, | 6.6 | (184) | 22.3 | (443) *** |
| % (N) | | | | |
| Myocardial | 6.7 | (189) | 1.8 | (37) *** |
| Infarction, % (N) | | | | |
| Heart Failure, % (N) | 1.3 | (37) | 1.6 | (33) |
| Stroke, % (N) | 3.6 | (102) | 1.3 | (26) *** |
| Sleep Disordered | 44.0 | (1234) | 62.1 | (1244) *** |
| Breathing, % (N) | | | | |
| Alcohol Use, % (N) | 45.7 | (1283) | 43.3 | (863) |
| Current Smoker, | 9.4 | (263) | 6.6 | (130) ** |
| % (N) | | | | |
| Anti-hypertensive | 35.5 | (996) | 52.9 | (1061) *** |
| Agent, % (N) | | | | |
| Anti-lipid Agent, | 11.3 | (316) | 37.0 | (742) *** |
| % (N) | | | | |
| Anti-hyperglycemic | 4.3 | (122) | 15.9 | (319) *** |
| Agent, % (N) | | | | |

| | | |
|---|---|---|
| Body Mass Index, | 28.2 ± 4.9 | 28.6 ± 5.5 ** |
| kg/m2 | | |
| Systolic Blood | 125.5 ± 18 | 122.9 ± 20 *** |
| Pressure, mmHg | | |
| Diastolic Blood | 73.2 ± 10.7 | 68.5 ± 9.9 *** |
| Pressure, mmHg | | |
| Non-HDL Cholesterol, | 155.4 ± 38 | 128.5 ± 36 *** |
| mg/dL | | |
| Framingham Risk, | 0.6 ± 1.1 | 0.8 ± 0.8 *** |
| score | | |
| CHA₂DS₂-VASc, | 2 ± 1 | 2 ± 1 *** |
| score | | |
| CHARGE-AF, score | 12.2 ± 1.3 | 12.9 ± 1.1 *** |
| DeepEntropy, units | 5.0 ± 1.7 | 4.3 ± 1.5 *** |
| CAFS, score | 4.5 ± 1.5 | 5.1 ± 1.4 *** |

Values are mean ± SD for continous variables and % (N) for binary variables
*p < 0.05
**p < 0.005
***p < 0.0001

The calculated FRS 10-year risk of myocardial infarction or death was also higher in the validation cohort (mean ±SD: 9.3±7.3 vs. 8.7±7.3%). Because both cohorts had a similar proportion of females (53 vs. 55%) but the validation cohort had older participants (68±9 vs. 63±11 years), age was considered the primary driver for the inflated values of the risk scores in the validation cohort.

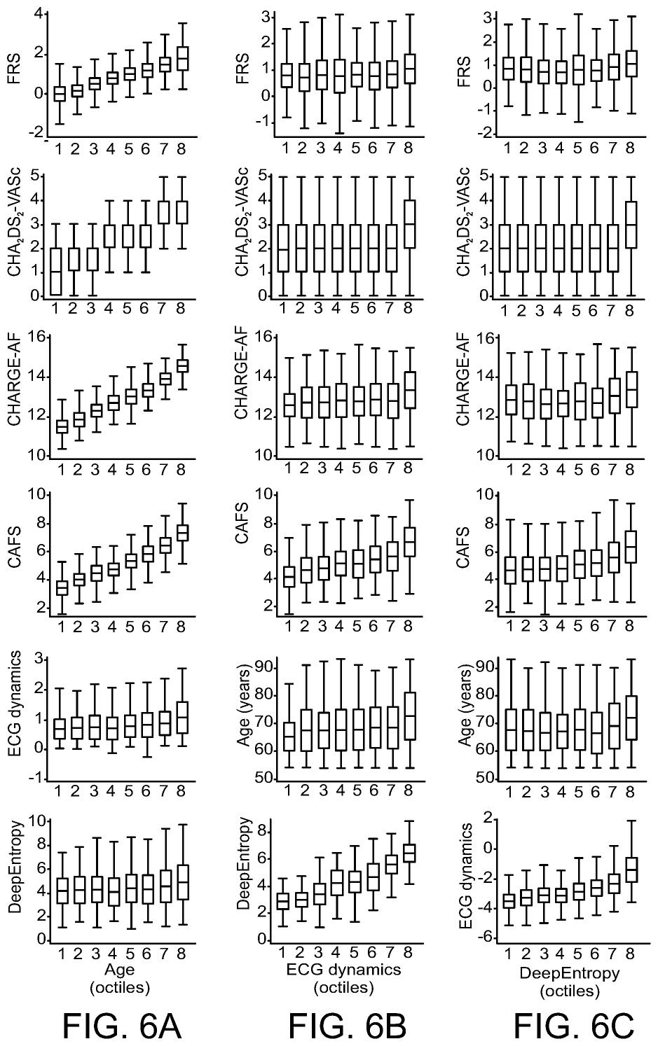

A correspondence assessment revealed strong correlation between age and each risk score, as demonstrated in FIGS. 6A-C. The plots show the median (horizontal line), 25-75% interquartile range (box) and range (whiskers) for each predictor vs. age (FIG. 6A), ECG dynamics (FIG. 6B) and DeepEntropy (FIG. 6C) in octiles. In particular, in FIG. 6A, it can be noted that, whereas age strongly correlated with a higher risk using CHARGE-AF (Spearman ρ=0.90), DeepEntropy calculated risk independent of age (Spearman ρ=0.085; P<0.0001). Whereas the conventional risk scores were directly associated with age, the ECG dynamics and DeepEntropy measures were completely independent of age and the conventional risk scores.

DeepEntropy values were also lower in the validation cohort (4.3±1.5 vs. 5.0±1.7), in accordance with the lower incidence of AF and cardiovascular events in the validation cohort. These results suggest that DeepEntropy, and indirectly CAFS, which incorporates DeepEntropy, will exhibit robust prognostic performance over broad and diverse populations.

The validation cohort provides an ideal opportunity to test this hypothesis. Compared to the derivation cohort, as demonstrated in Table 1, the validation cohort is more racially diverse, e.g., African Americans (28 vs. 7%), Hispanics (24 vs. 7%), Chinese Americans (12 vs. 0%). In the validation cohort, hypertension was more prevalent (56 vs. 37%), but the use of anti-hypertensive medications was also greater (53 vs. 35%), and both the systolic and diastolic blood pressures were lower (123/68 vs. 126/73 mmHg). The use of anti-lipid agents was also three times greater, and non-HDL cholesterol levels were lower (128±36 vs. 155±38 mmHg). The prevalence of diabetes was four times greater, along with four times greater use of anti-glycemic agents, and HbA1c levels were 6.0±0.9. In addition to greater control of cardiovascular risk in the validation cohort, the participants did not have a history of advanced cardiovascular disease. By contrast, those in derivation cohort had preexisting myocardial infarction (7%), heart failure (1%), and stroke (4%).

The above comparison of the derivation and validation cohorts highlights the dominant impact of age in the calculation of FRS, CHA₂DS₂-VASc and CHARGE-AF. Moreover, they have significant overlap in predictors with each other as well as with other generalized risk scores such as atherosclerosis. By contrast, DeepEntropy proved to be a fundamentally distinct measure from these risk scores in the derivation cohort, and predicted AF incidence independent of a comprehensive set of conventional risk factors. Thus, it follows that CAFS, which incorporates DeepEntropy and conventional risk factors, would perform better than the FRS, CHA₂DS₂-VASc, and CHARGE-AF scores at predicting AF incidence across an older, more diverse population with greater control of cardiovascular risk (e.g., from medications) that more closely represents patients likely to experience AF.

### Example 3

### Predictors of AF Incidence in the Validation Cohort

Over a follow-up period of 8.3±0.89 years, 84 out of 2,004 participants developed new-onset AF. As demonstrated in Table 2, those who developed AF were more likely to be older and Caucasian; have a history of hypertension, lower non-HDL-cholesterol levels, and lower diastolic blood pressure; have higher values for the FRS, CHA₂DS₂-VASc, CHARGE-AF, DeepEntropy (2.9±2.6 vs. 1.3±1.5) and CAFS.

TABLE 2

| Covariate | Incident AF | | No Incident AF | | Total Cohort | |
|---|---|---|---|---|---|---|
| Age, years | 73.6 ± 8.8 | | 68.1 ± 9.1 *** | | 68.4 ± 9.1 | |
| Female, % (N) | 46.3 | (38) | 53.6 | (1031) | 1069 | (53.3) |
| White, % (N) | 54.9 | (45) | 35.5 | (683) ** | 728 | (36.3) |
| Chinese, % (N) | 11.0 | (9) | 12.1 | (232) | 241 | (12) |
| Black, % (N) | 22.0 | (18) | 28.1 | (541) | 559 | (27.9) |
| Hypertension, % (N) | 71.6 | (58) | 55.8 | (1072) ** | 1130 | (56.4) |
| Diabetes Mellitus, | 22.5 | (18) | 22.3 | (425) | 443 | (22.3) |
| % (N) | | | | | | |
| Myocardial | 2.4 | (2) | 1.8 | (35) | 37 | (1.8) |
| Infarction, % (N) | | | | | | |
| Heart Failure, % (N) | 12.2 | (10) | 1.2 | (23) *** | 33 | (1.6) |
| Stroke, % (N) | 2.4 | (2) | 1.2 | (24) | 26 | (1.3) |
| Sleep Disordered | 63.4 | (52) | 62.0 | (1192) | 1244 | (62.1) |
| Breathing, % (N) | | | | | | |
| Alcohol Use, % (N) | 43.9 | (36) | 43.3 | (827) | 863 | (43.3) |
| Current Smoker, | 3.7 | (8) | 6.7 | (127) | 130 | (6.6) |
| % (N) | | | | | | |
| Former Smoker, % (N) | 41.5 | (34) | 38.0 | (721) | 755 | (38.2) |
| Never Smoker, % (N) | 54.9 | (45) | 55.3 | (1049) | 1094 | (55.3) |
| Anti-lipid Agent, | 50.0 | (41) | 36.5 | (701) * | 742 | (37) |
| % (N) | | | | | | |
| Aspirin, % (N) | 52.4 | (43) | 42.7 | (820) | 863 | (43.1) |
| Insulin, % (N) | 7.3 | (6) | 2.9 | (56) * | 62 | (3.1) |
| Oral Anti- | 12.2 | (10) | 14.7 | (283) | 293 | (14.6) |
| hyperglycemic | | | | | | |
| Agent, % (N) | | | | | | |
| Oral Anticoagulant, | 26.8 | (22) | 0.7 | (14) *** | 36 | (1.8) |
| % (N) | | | | | | |
| Anti-hypertensive, | 81.7 | (67) | 51.7 | (994) *** | 1061 | (52.9) |
| % (N) | | | | | | |
| Atrioventricular | 32.1 | (26) | 20.4 | (389) * | 415 | (20.9) |
| conduction delay, | | | | | | |
| % (N) | | | | | | |

| | | | |
|---|---|---|---|
| Body Mass Index, | 28.8 ± 5.8 | 28.6 ± 5.5 | 28.6 ± 5.5 |
| kg/m2 | | | |
| Systolic Pressure, | 122.0 ± 17.5 | 122.9 ± 20.3 | 122.9 ± 20.3 |
| mmHg | | | |
| Diastolic Pressure, | 66.3 ± 10.6 | 68.6 ± 9.8 * | 68.5 ± 9.9 |
| mmHg | | | |
| Non-HDL Cholesterol, | 109.3 ± 35.1 | 129.3 ± 35.6 *** | 128.5 ± 35.8 |
| mg/dL | | | |
| Cholesterol, mg/dL | 164.3 ± 39.9 | 184.8 ± 36.3 *** | 184.0 ± 36.7 |
| Triglyceride, mg/dL | 111.9 ± 87.5 | 110.6 ± 63.8 | 110.7 ± 64.9 |
| High Density | 55.0 ± 16.4 | 55.6 ± 16.3 | 55.5 ± 16.3 |
| Lipoprotein, mg/dL | | | |
| FRS, score | 1.1 ± 0.9 | 0.8 ± 0.8 ** | 0.8 ± 0.8 |
| CHA₂DS₂-VASc, | 3 ± 1 | 2 ± 1 *** | 2 ± 1 |
| score | | | |
| CHARGE-AF, score | 13.6 ± 1.1 | 12.8 ± 1.1 *** | 12.9 ± 1.1 |
| DeepEntropy, units | 5.1 ± 1.7 | 4.3 ± 1.5 *** | 4.3 ± 1.5 |
| CAFS, score | 6.4 ± 1.8 | 5.1 ± 1.4 *** | 5.1 ± 1.4 |

Values are mean ± SD for continous variables and % (N) for binary variables
*p < 0.05
**p < 0.005
***p < 0.0001

Whereas non-Caucasians were at a higher risk for developing AF in the derivation cohort (11.4 vs. 6.9%), Caucasians have a higher risk in the validation cohort (54.9 vs. 35.5%). Interestingly, low non-HDL-cholesterol was significantly associated with AF incidence in both cohorts. A comprehensive screen for correlating variables yielded no confounders, including medication use. Some of the conventional predictors that had shown strong, independent predictive value in the derivation cohort were either less significant or not significant in the validation cohort (e.g., diastolic blood pressure, history of diabetes, social habits such as smoking and alcohol use). Greater use of medications in the validation cohort may account for these discrepancies.

### Example 4

### Comparison of Model Performance in the Validation Cohort

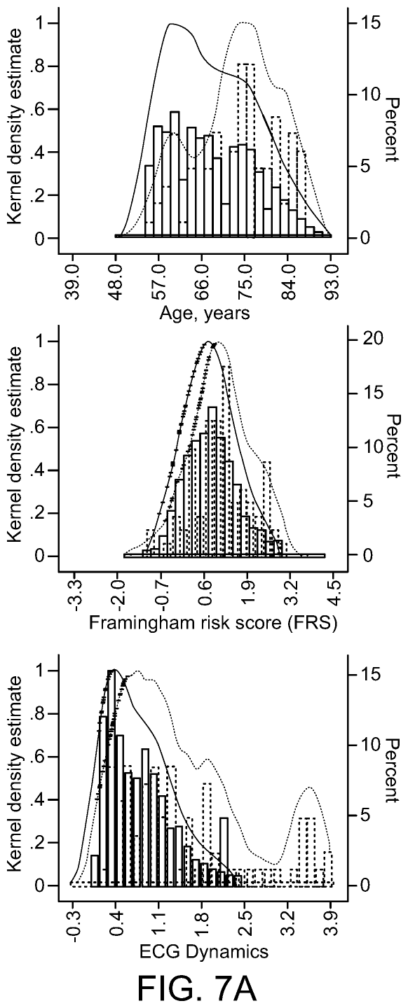

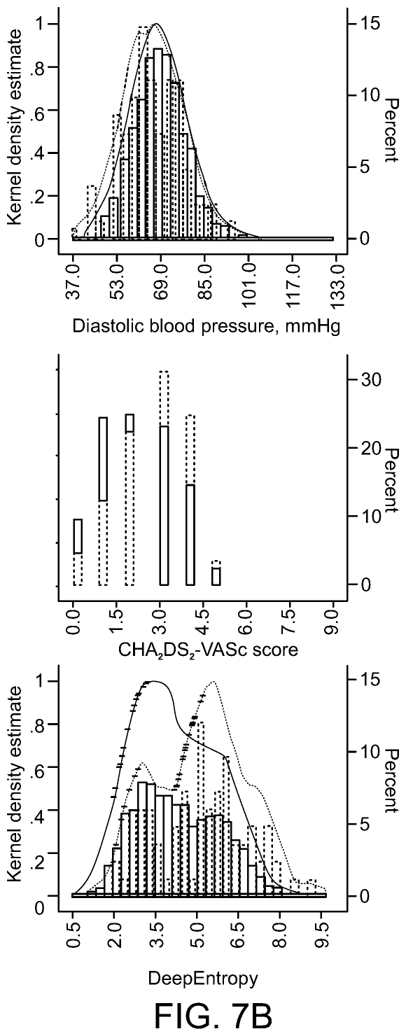

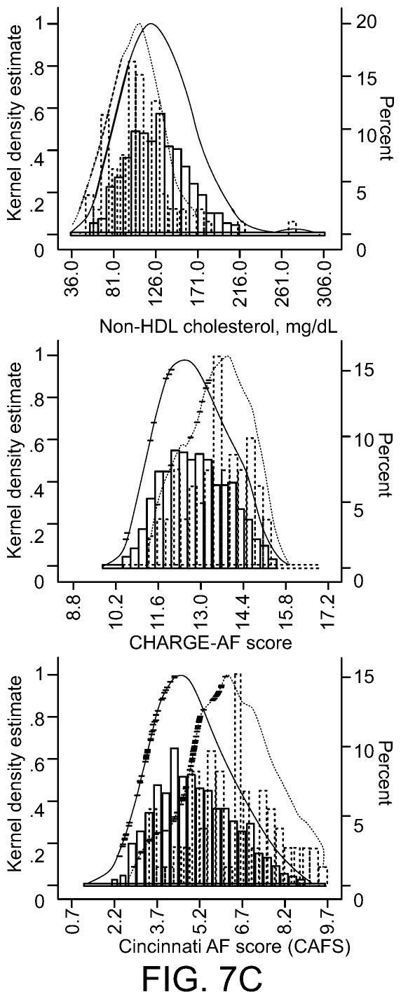

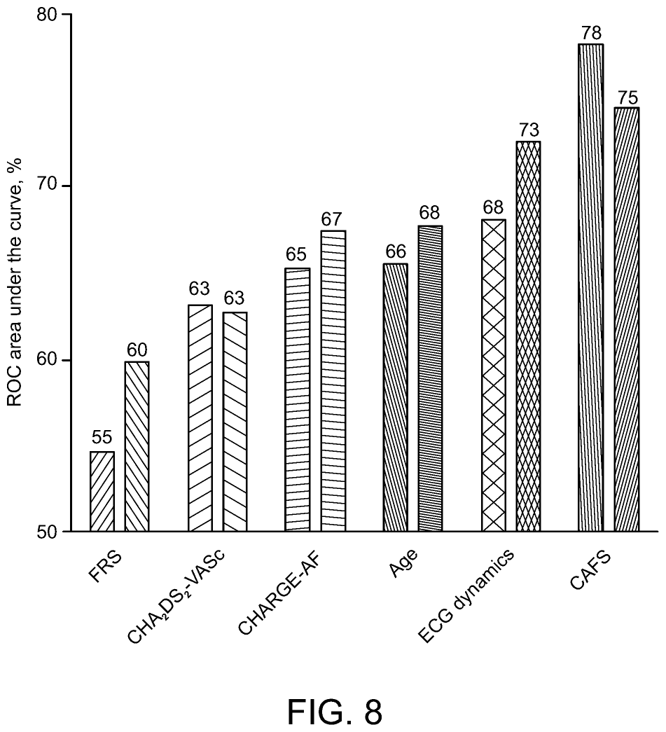

FIGS. 7A-7C depict a comparison of distributions, probability densities and ROC curves between key predictors and risk scores. The histograms are superimposed with non-parametric kernel density plots for groups who developed AF and did not develop AF over the follow-up period. As demonstrated in FIG. 8, the ROC area under the curve (AUC) at 6 and 10 years of follow-up were greater for CAFS compared to the FRS, CHA₂DS₂-VASc, CHARGE-AF, age, and ECG Dynamics. The corresponding odds ratios for the third compared to first tertiles (t3:t1) of age, FRS, CHA₂DS₂-VASc, CHARGE-AF, DeepEntropy, and CAFS were 1.2 [95% Cl, 0.53-1.84] 2.3 [95% Cl, 1.3-4.2], 3.2 [1.6-6.4], 6.1 [2.4-15.5], 8.1 [4.6-14.3], and 8.2 [3.2-20.4], respectively.

ECG Dynamics and CAFS consistently performed better than age, FRS, CHA₂DS₂-VASc score, and CHARGE-AF score for predicting the incidence of AF as can be seen in FIG. 8. Interestingly, age and CHARGE-AF had similar C-statistic values, which were greater than the FRS and CHA₂DS₂-VASc scores. DeepEntropy alone exhibited more predictive value than the other conventional risk scores. Notably, removing all predictors from CAFS except for age and DeepEntropy yielded the same C-statistic (0.75) as in the derivation cohort.

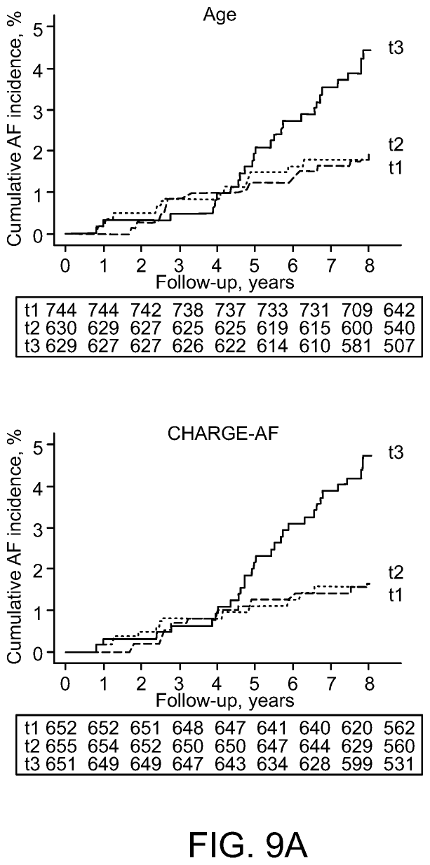

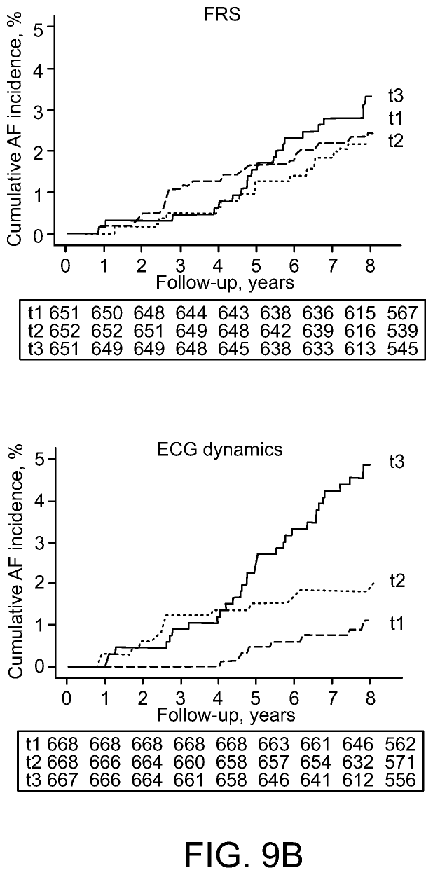

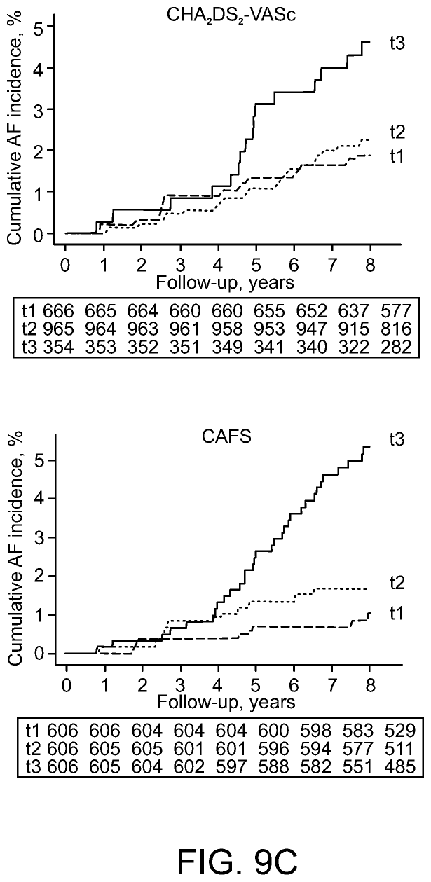

FIGS. 9A-9C depict the cumulative incidence of atrial fibrillation, plotted by tertiles of each risk score or predictor for a follow-up of 8 years. The corresponding number at risk table is shown below each plot. The hazard ratios for the third tertile compared to the first tertile (t3:t1) of age, FRS, CHA₂DS₂-VASc, CHARGE-AF, ECG dyna mics, and CAFS were 3.6 [95% CI, 1.8-6.4], 2.3 [1.3-4.2], 3.4 [1.7-6.5], 6.5 [2.6-16.3], 7.3 [4.2-12.4] and 8.0 [3.2-20.0], respectively. In Cox proportional hazards analyses, the adjusted hazard ratios were highest for DeepEntropy and CAFS. Again, the cumulative incident plot for age was nearly identical to that of CHARGE-AF, suggesting age is its primary driver.

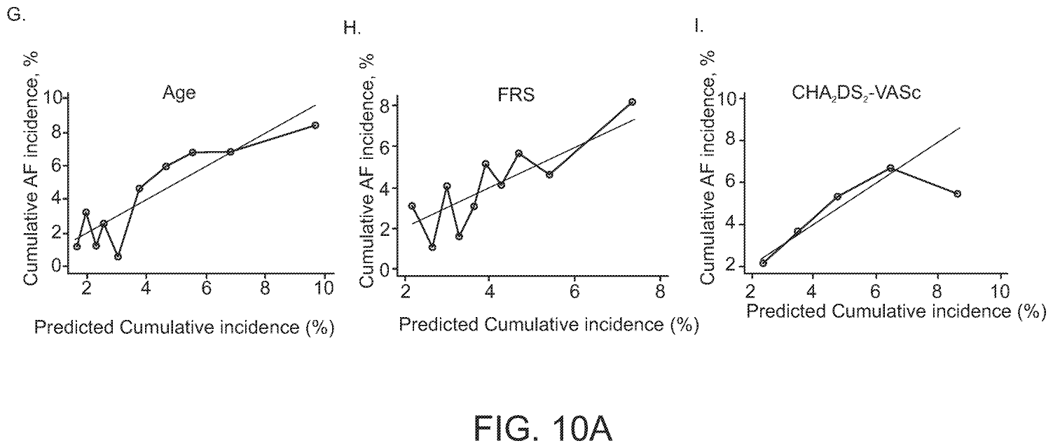

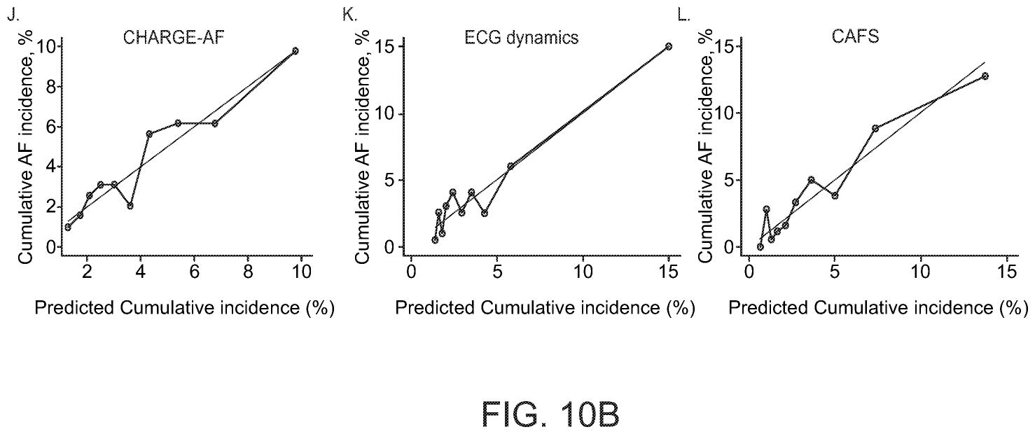

FIGS. 10A-10C depict Hosmer-Lemeshow calibration curves for the plots of FIGS. 9A-9C. The calibration curves were used to compare the predicted and observed risk for cumulative AF incidence within each subgroup defined by either score. A shorter distance from the connected round markers to the 45° reference line indicates better calibration. All risk scores were well-calibrated based on the Hosmer-Lemeshow test. DeepEntropy exhibited the best calibration (X2=0.66; P=0.99), followed by CHA₂DS₂-VASc (X2=1.32; P=0.93), CHARGE-AF (X2=3.07; P=0.98), CAFS (X2=5.05; P=0.89), FRS (X2=7.23; P=0.70) and age (X2=10.15; P=0.43).

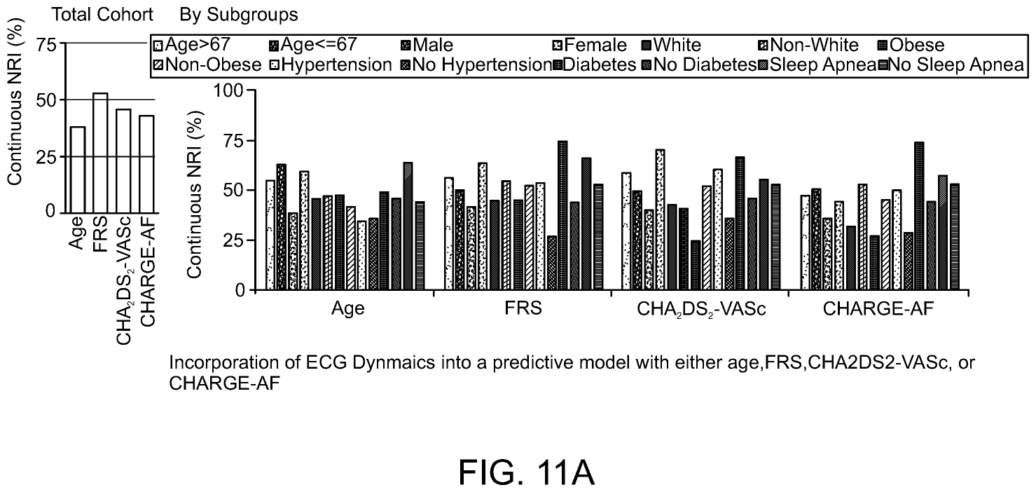

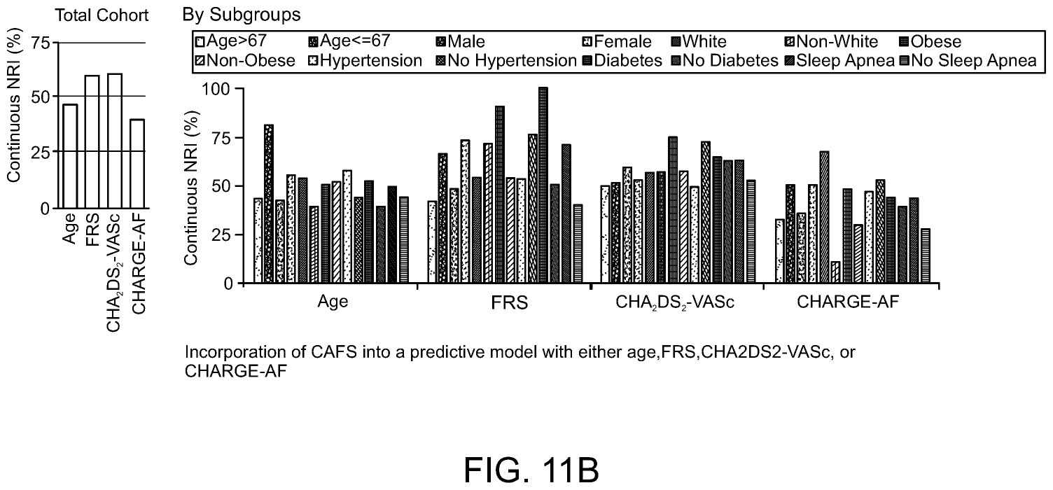

In a predicative model comprised of age, FRS, CHA₂DS₂-VASc, or CHARGE-AF score, incorporating either ECG dynamics, as shown in FIG. 11A or CAFS, as shown in FIG. 11B improved net reclassification. Subgroup analyses revealed consistent NRI values regardless of age, sex, and presence or absence of conventional risk factors.

Net reclassification improvement (NRI) after incorporating ECG dynamics to each model are shown for the entire cohort (left) and by subgroups of risk factors (right) of FIG. 11A. The corresponding integrated discrimination improvement (IDI) for the total cohort for age, FRS, CHA₂DS₂-VASc and CHARGE-AF were 5.3 [3.2-5.9], 6.3 [3.6-6.5], 5.8 [3.6-6.2] and 5.7 [3.5-5.9] %, respectively. The NRI after incorporating CAFS to each model are shown for the entire cohort (left) and by subgroups of conventional risk factors (right) of FIG. 11B. There was no evidence of interactions between DeepEntropy with any of these risk factors. These results are consistent with the findings in the derivation cohort.

### Example 5

### Modified Cohort Analysis

To reduce any confounders due to the different participant demographics between the validation and derivation cohorts, extensive modified cohort analysis was performed, all of which, yielded CAFS to be a superior model compared to FRS, CHA₂DS₂-VASc and CHARGE-AF.

Individuals with missing follow-up ECG, presence of prior AF, and poor quality ECG tracings were excluded. Those included (N=2807) and excluded (N=2788) were similar in age (mean±SD: 62.6±11 in inclusion cohort vs 63.3±11 in exclusion cohort) and BMI. The inclusion cohorts had more females (55 vs 50%) and the exclusion group had a significantly higher prevalence of heart failure, diabetes mellitus, and hypertension. Increased prevalence of comorbidities correlated with the higher risk score values, FRS, CHA₂DS₂-VASc and CHARGE-AF, in the exclusion cohort.

However, analyses performed by substituting the population mean for missing data, censoring people with missing data, or 10-fold cross-validation yielded similar results, suggesting that the missing data had no significant effect on our findings. Moreover, individuals with paroxysmal asymptomatic AF may have been misclassified as non-cases in this validation study because some diagnoses of AF were self-reported. Nevertheless, the two, separate, comprehensive, prospective, multicenter, derivation and validation studies in community adults with about 10 years of follow-up is one of the greatest strengths of the present disclosure. These two very different cohorts evidence the vast application of DeepEntropy and CAFS in a diverse population and provide strong supportive evidence for their clinical application.

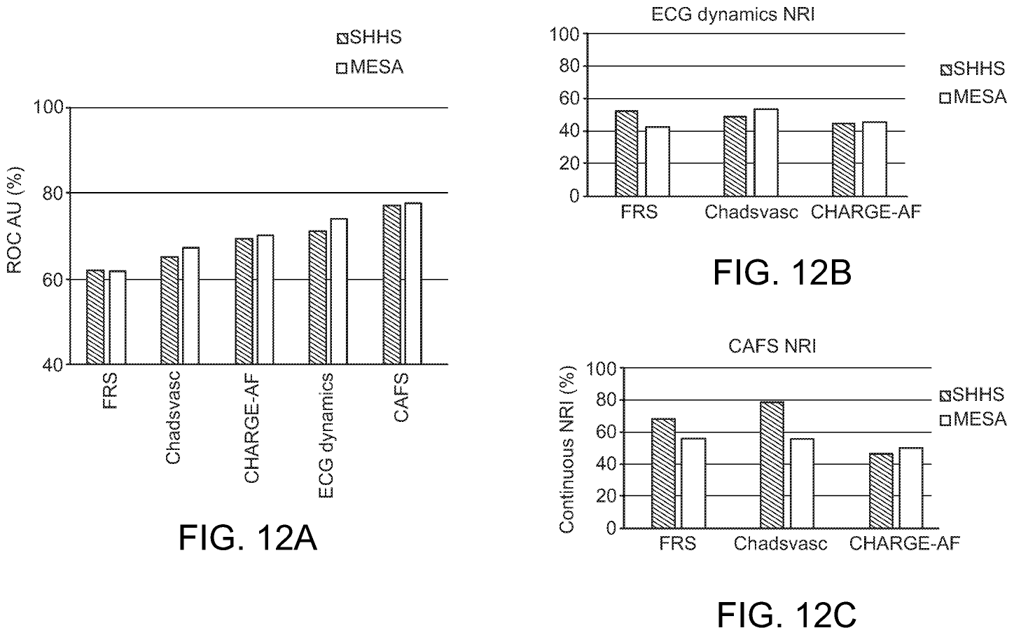

Matched subgroups from both the validation and derivation cohorts were created based on age, AF incidence, and race. Data from this is shown in Table 3 and FIGS. 12A-12C, demonstrating CAFS to be a superior model compared to FRS, CHA₂DS₂-VASc and CHARGE-AF.

TABLE 3

| | | |
|---|---|---|
| | M-Derivation | M-Validation |
| Covariate | (N = 909) | (N = 909) |

| | | |
|---|---|---|
| Age, years | 68.0 ± 8.5 | 68.0 ± 8.5 |

| | | | | |
|---|---|---|---|---|
| Female, % (N) | 53.5 | (486) | 53.7 | (488) |
| Caucasian, % (N) | 77.4 | (704) | 77.4 | (704) |
| African American, | 15.6 | (142) | 15.6 | (142) |
| % (N) | | | | |
| History of | 50.8 | (461) | 44.1 | (401) ** |
| Hypertension, % (N) | | | | |
| Incident AF, % (N) | 5.7 | (52) | 5.7 | (52) |

| | | |
|---|---|---|
| Framingham Risk | 0.8 ± 0.8 | 1.1 ± 0.8 *** |
| Score, score | | |
| CHA2DS2-VASc, score | 2.1 ± 1.3 | 2.1 ± 1.4 |
| CHARGE-AF, score | 12.8 ± 1.0 | 12.8 ± 1.0 |
| Complexity of sinus | 1.2 ± 1.5 | 1.7 ±14 *** |
| rhythm, DeepEntropy | | |
| CAFS, score | 5.0 ± 1.3 | 5.2 ± 1.2 |

* < 0.05,
** < 0.001,
*** < 0.0001

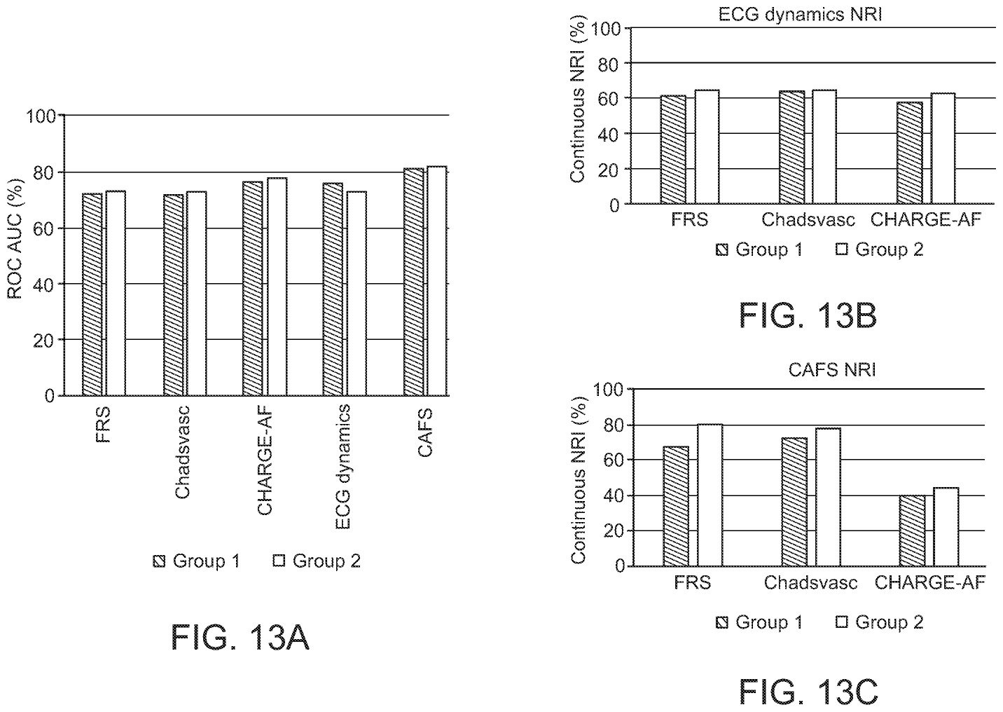

Randomly matched cohorts based on age, AF incidence, and race were selected with participants from both cohorts. Data from this analysis is presented in Table 4 and FIGS. 13A-13C, once again demonstrating CAFS to be a superior model compared to FRS, CHA₂DS₂-VASc and CHARGE-AF.

TABLE 4

| | Group 1 | Group 0 |
|---|---|---|
| Covariate | (2364) | (N = 2439) |

| | | |
|---|---|---|
| Age, years | 65.0 ± 10.8 | 65.0 ± 10.8 |

| | | | | |
|---|---|---|---|---|
| Female, % (N) | 54.1 | (1324) | 54.2 | (1282) |
| Caucasian, % (N) | 64.7 | (1583) | 65.7 | (1552) |
| African American, | 16.2 | (396) | 15.7 | (371) |
| % (N) | | | | |
| History of | 46.0 | (1126) | 44.5 | (1052) |
| Hypertension, % (N) | | | | |
| Incident AF, % (N) | 8.7 | (213) | 7.8 | (184) |

| | | |
|---|---|---|
| Framingham Risk | 0.7 ± 1.0 | 0.7 ± 1.0 |
| Score, score | | |
| CHA2DS2-VASc, score | 2.0 ± 1.4 | 1.9 ± 1.4 |
| CHARGE-AF, score | 12.5 ± 1.3 | 12.5 ± 1.3 |
| Complexity of sinus | 1.5 ± 1.5 | 1.5 ± 1.5 |
| rhythm, DeepEntropy | | |
| CAFS, score | 4.8 ± 1.5 | 4.8 ± 1.5 |

* < 0.05,
** < 0.001,
*** < 0.0001

### Example 6

### Sleep Disordered Breathing

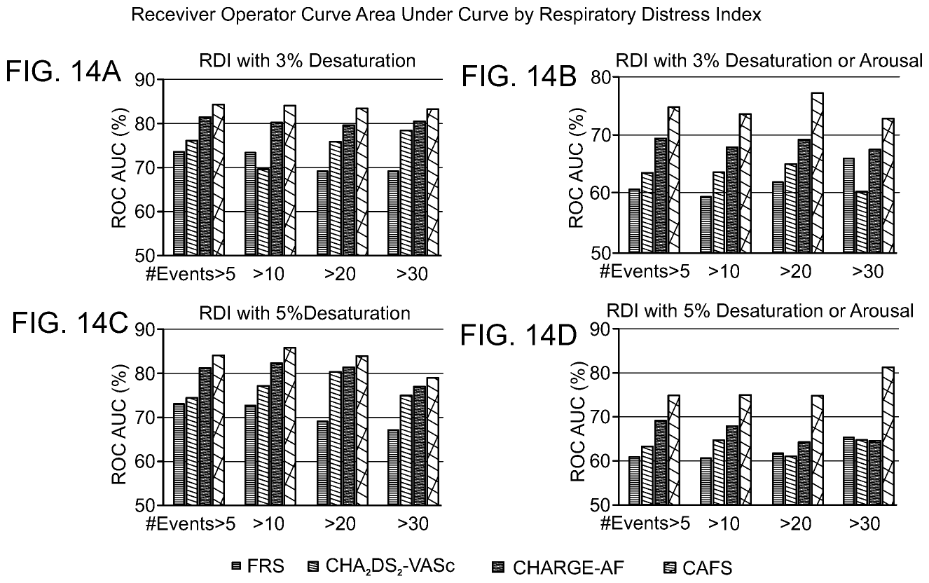

In the validation cohort, comprised of racially diverse, community adults without existing cardiovascular disease, dynamic ECG measures during sleep strongly predicted AF incidence over follow-up. These dynamic ECG measures are fundamentally distinct from established AF risk scores and contribute unique prognostic insight, regardless of the absence or presence of conventional risk factors, including varying degree of sleep disordered breathing, as shown in FIGS. 14A-14D. These results confirm that CAFS, consistently outperforms the FRS, CHA₂DS₂-VASc and CHARGE-AF risk scores for predicting AF incidence. This paradigm for improved identification of who will or will not develop AF has important implications for preventing AF associated complications as well as AF itself.

### Example 7

### Complexity of Sinus Rhythm

Time, frequency, and non-linear domain analyses of HRV was performed by 30- and 300-second binned epochs and stratified by the following sleep states: (1) awake just before onset of sleep, (2) immediately after sleep onset, (3) rapid eye movement (REM), (4) non-REM, (5) transient awakenings during sleep, (6) just before waking up from sleep, and (7) at or immediately after waking up from sleep.

These state-specific analyses were not performed in bins longer than 300 seconds to eschew noisy data or introducing bias from missing data (e.g., only 64% of participants had adequate quality ECG recordings five minutes after waking from sleep). Independent of sleep state, 1-hour binned analyses (e.g., first hour of sleep, second hour, third hour, etc.) were performed. In addition to performing non-linear dynamic analysis of the 30-sec, 300-sec, and 1-hour binned epochs with an extensive set of established algorithms (e.g., approximate entropy, detrended fluctuation analysis, sample entropy, multi-scale sample entropy, EntropyX), entropy estimation and machine learning were also incorporated to more accurately extract the nonlinear dynamics of sinus rhythm from the longer, 1-hour binned epochs using DeepEntropy.

DeepEntropy quantifies the degree to which subtle patterns of self-similar fluctuations in sinus rhythm repeat themselves over an hour. These non-linear patterns are typically beyond human cognition and analytical synthesis, but can be revealed using DeepEntropy (values ranging from 0 to 8). Lower values of DeepEntropy reflecting more self-similar fluctuations imply increased coupling of periodic processes among organ systems that typically are a signature of health, such as respiratory sinus arrhythmia. By contrast, higher values of DeepEntropy imply reduced repetition of older patters and increased generation of newer patterns underlying the variability of sinus rhythm, implying progressive uncoupling of homeostatic processes. Unlike conventional measures of HRV, DeepEntropy analysis of a time series does not require equally sampled time intervals or extensive preprocessing of data. Unlike approximate entropy, sample entropy, frequency domain measures of H RV, or geometric measures such as Poincaré plots, DeepEntropy measures are insensitive to the degree of tolerance allowed for matching templates and less sensitive to the presence of outlying points, such as ectopic beats or noise. Unlike approximate entropy, sample entropy, or EntropyX, DeepEntropy can be calculated for long time series (e.g., hours).

### Example 8

### Comparison of CAFS with Established Risk Scores for Predicting New-Onset AF

The distribution of values of CAFS were more distinct between individuals who developed and did not develop AF compared to CHARGE-AF, CHA₂DS₂-VASc, and FRS. The ROC curve area for CAFS (85.8%) was higher than that of FRS (75.9%), CHA₂DS₂-VASc (78.1%) and CHARGE-AF (82.9%) (FIG. 2A). The superior performance of CAFS was driven primarily by the dynamic ECG measures, both in the presence and absence of hypertension. In subgroup analyses, CAFS outperformed FRS, CHA₂DS₂-VASc and CHARGE-AF for predicting new-onset AF, regardless of age, race, gender, BMI and the presence of other cardiovascular risk factors.

In subgroup analyses, CAFS outperformed FRS, CHA₂DS₂-VASc, and CHARGE-AF for predicting new-onset AF, regardless of age, race, gender, BMI and the presence of other cardiovascular risk factors. The performance of CAFS was greatest in those who are older, had hypertension, and had sleep disordered breathing, yielding ROC curve areas of 15%, 13%, and 11% greater than FRS, respectively. More specifically, CAFS performed better than FRS, CHA₂DS₂-VASc and CHARGE-AF over a broad range of ages and degrees of sleep disordered breathing consistent with the poor correlation of DeepEntropy with age and SDB. Indeed, DeepEntropy had little or no correlation with all conventional clinical predictors and PSG parameters.

Over a mean follow-up of 5.3±0.29 years, 315 (11.2%) out of 2,807 participants in the derivation cohort developed new-onset AF. Those who developed AF were more likely to be older; have lower diastolic blood pressure and lower non-HDL cholesterol; and were more likely to have a history of hypertension, diabetes mellitus, congestive heart failure, myocardial infarction and SDB, including having a higher apnea-hypopnea incidence and spend a larger proportion of their sleep time in apnea. There was no association with alcohol consumption. As expected, the FRS, CHA₂DS₂-VASc, and CHARGE-AF risk scores were significantly greater in those who developed AF.

In univariate analysis of the derivation cohort, the most significant conventional predictors of AF included older age, black race/ethnicity, history of hypertension, history of myocardial infarction, history of congestive heart failure (or prescribed digoxin), lower values of total and non-HDL cholesterol, reduced FEV1, and reduced FVC. The most significant PSG parameters included higher arousal index, RDIs for all levels of oxygen desaturations with and without arousal, and central and obstructive apnea index, and lower oxygen saturation during REM and non-REM sleep, and apnea-hypopnea index.

BMI was not a significant predictor of new-onset AF (p=0.82). CHARGE-AF was a stronger predictor of new-onset AF compared to the FRS and CHA₂DS₂-VASc risk score. The dynamic ECG measures strongly predicted new-onset AF, including (1) blunted chronotropic response during transient mild hypoxemia, (2) an increase in the high frequency component of the heart rate power spectral density reflecting primarily parasympathetic tone during early waking hours, (3) subtle atrioventricular or intraventricular conduction abnormality excluding complete left bundle branch block and right bundle branch block, and (4) non-linear dynamic analysis of sinus rhythm complexity during the first hour of sleep onset, as indexed by DeepEntropy. For example, the odds of AF incidence is 2.31 times greater in those in the 3rd tertile of DeepEntropy compared to those in the 1st tertile, which equates to 0.71 risk of AF incidence.

In the multicenter, racially diverse, community cohort comprised of adults without advanced cardiovascular disease, the established scores for predicting AF incidence performed poorly, due to their considerable overlap with generalized risk factors such as atherosclerotic burden, reliance on medical history, and the strong influence of age. By contrast, CAFS incorporates DeepEntropy, a fundamentally distinct measure that reflects subclinical pathogenic mechanisms, namely arrhythmogenic substrates and triggers, as well as modulating factors and their interactions for personalized prediction of AF incidence.

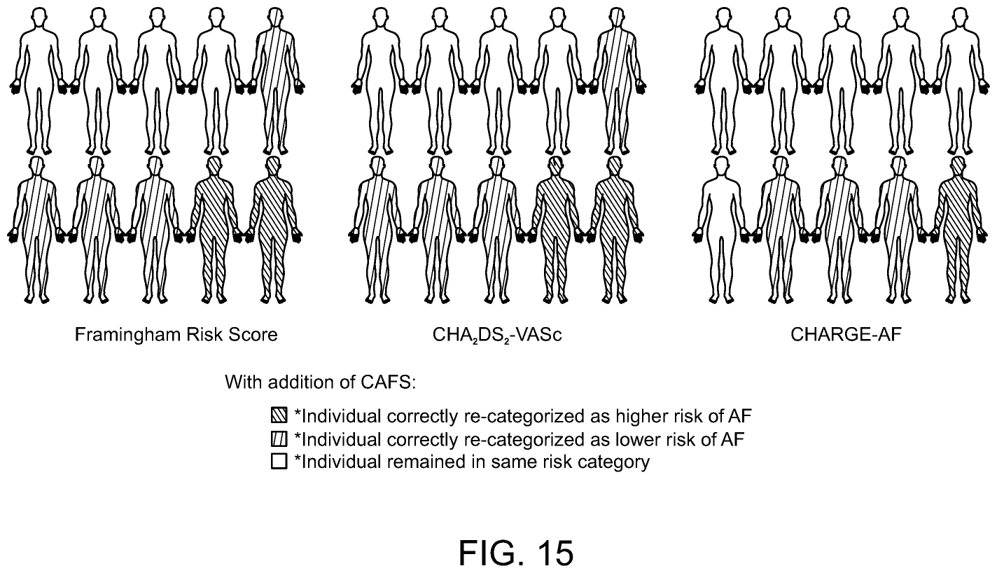

This new paradigm validated herein for detecting early, subclinical signatures of physiological deterioration has potential to expand the clinical utility of PSG beyond diagnosing SDB and yielding broad and meaningful clinical impact. As demonstrated in FIG. 15, incorporating dynamic ECG analysis in a multivariate model comprised of the conventional risk scores improved both true positives and true negatives.

All documents cited are incorporated herein by reference; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

The foregoing description is illustrative of particular embodiments of the invention but is not meant to be a limitation upon the practice thereof. While particular embodiments have been illustrated and described, it would be obvious to one skilled in the art that various other changes and modifications can be made without departing from the spirit and scope of the invention. It is therefore intended to cover in the appended claims all such changes and modifications that are within the scope of this invention.

1. A method for diagnosing and treating a subject at risk for atrial fibrillation (AF) or AF-related health conditions, the method comprising:
collecting one or more physiological signals from the subject in a sleep state or an awake state;
extracting time series data from the one or more physiological signals;
performing dynamic analyses of the time series data using artificial intelligence, wherein the artificial intelligence calculates a series of dynamic measurements, said dynamic measurements being indicative of a probability of an onset of an abnormal atrial rhythm;
providing an integrated personalized risk score comprising the dynamic measurements, wherein the integrated personalized risk score is indicative of a probability of an onset of AF in the subject;
diagnosing the subject as being at risk for AF when the integrated personalized risk score exceeds a threshold value, wherein the threshold value is calculated by the artificial intelligence based on a library of stored data; and
treating the diagnosed subject with an effective therapy to prevent or treat AF or AF-related health conditions.

2. The method of claim 1, wherein the one or more physiological signals are selected from the group consisting of electrocardiogram (ECG), phonocardiograms (PCG), photoplethysmogram (PPG), intra-aortic balloon pump (IABP), respiratory frequency signal prominence (RSP), transmembrane potentials (TMP), ultrasound guided (USG), and audio compression manager (ACM) waveforms.

3. The method of claim 1, wherein the one or more physiological signals are collected while the subject is asleep, before the subject falls asleep, or after the subject wakes from sleep.

4. The method of claim 1, wherein the one or more physiological signals is associated with the sleep state.

5. The method of claim 1, wherein the artificial intelligence generates the dynamic measurements by applying a DeepEntropy calculation to the time series data to generate the probability of the onset of the abnormal atrial rhythm.

6. The method of claim 5, wherein the dynamic analyses generate an index of a complexity of sinoatrial and atrioventricular function in the subject.

7. The method of claim 5, wherein the dynamic analyses are performed to generate an index of intraatrial and atrioventricular conduction abnormality in the subject.

8. The method of claim 5, wherein the dynamic analyses are performed to generate an index based on a measure of heart rate variability while the subject is transitioning from the sleep state to the awake state and wherein the subject is fully awake for a period of at least 5 minutes.

9. The method of claim 1, wherein the integrated personalized risk score is also based on one or more additional risk factors.

10. The method of claim 9, wherein the one or more additional risk factors are selected from the group consisting of: sex; age; body mass index; history of hypertension; blood pressure; cholesterol; incidence of previous heart problems; diabetes; smoking status; race; and combinations thereof.

11. The method of claim 9, wherein the one or more additional risk factors is selected from the group consisting of chronotropic response during transient mild hypoxemia; subtle atrioventricular and intraventricular conduction dynamics; a high frequency component of ECG power spectral density, and combinations thereof.

12. The method of claim 11, wherein the one or more additional risk factors is measured while the subject is asleep.

13. The method of claim 1, wherein the therapy to prevent or terminate AF is selected from the group consisting of: life style changes, cognitive-behavioral therapy, biofeedback therapy, vasovagal maneuvers, autonomic modulation, electrical cardioversion, electrical pacing, pharmacological therapy, ablation, surgery, and combinations thereof.

14. The method of claim 13, wherein the therapy to prevent or terminate AF is pharmacological therapy selected from the group consisting of beta blockers, calcium channel blockers, digoxin, anti-arrhythmic medications, and anticoagulants.

15. The method of claim 1, further comprising collecting a plurality of physiological signals and performing dynamic analyses on each of the plurality of signals and combinations of the plurality of signals.

16. The method of claim 1, wherein dynamic measurements further indicate the probability of the abnormal atrial rhythm occurring within an acute time period, a subacute time period, or an extended time period.

17. The method of claim 1, wherein the threshold value is calculated using the library of data to determine a first tertile, a second tertile, and a third tertile, wherein a score in the third tertile corresponds to an increased probability of developing AF.

18. The method of claim 17, wherein the artificial intelligence uses the library of stored data to calculate varying threshold values based on different risk factors.

19. The method of claim 1, further comprising repeating collecting one or more physiological signals and performing dynamic analyses at one or more subsequent time points.

20. The method of claim 1, wherein the artificial intelligence responds to the integrated personalized risk score to determine a personalized treatment plan.
